SEQUENCE LISTING (11 sequences)

SEQ ID NO: 1 - protein, 15 aa, Artificial sequence
  CDR3 beta amino acid sequence
CASSQGGTTNSDYTF

SEQ ID NO: 2 - protein, 13 aa, Artificial sequence
  CDR3 beta amino acid sequence
CASSRDTNSDYTF

SEQ ID NO: 3 - protein, 14 aa, Artificial sequence
  CDR3 beta amino acid sequence
CASSQRGANTEVFF

SEQ ID NO: 4 - protein, 12 aa, Artificial sequence
  CDR3 beta amino acid sequence
CASSIRGTEVFF

SEQ ID NO: 5 - protein, 14 aa, Artificial sequence
  CDR3 beta amino acid sequence
CASSQDTNTGQLYF

SEQ ID NO: 6 - protein, 12 aa, Artificial sequence
  CDR3 beta amino acid sequence
CASSTGGDTQYF

SEQ ID NO: 7 - protein, 13 aa, Artificial sequence
  CDR3 beta amino acid sequence
CASSLLGSYEQYF

SEQ ID NO: 8 - protein, 12 aa, Artificial sequence
  CDR3 beta amino acid sequence
CASSIRGTEVFF

SEQ ID NO: 9 - protein, 12 aa, Artificial sequence
  CDR3 beta amino acid sequence
CASSTGGDTQYF

SEQ ID NO: 10 - protein, 15 aa, Artificial sequence
  CDR3 beta amino acid sequence
CASSQGGTTNSDYTF

SEQ ID NO: 11 - protein, 14 aa, Artificial sequence
  CDR3 beta amino acid sequence
CASSQDTNTGQLYF

(19) United States
(12) Patent Application Publication
(10) Pub. No.: US 2022/0265725 A1
(43) Pub. Date: Aug. 25, 2022

(54) USE OF VETO CELLS IN TREATMENT OF T CELL MEDIATED AUTOIMMUNE DISEASES

(71) Applicant: Yeda Research and Development Co. Ltd., Rehovot (IL)

(72) Inventors: Yair REISNER, Houston, TX (US); Rakefet SIDLIK MUSKATEL, Rehovot (IL); Bar NATHANSOHN-LEVI, Rehovot (IL)

(73) Assignee: Yeda Research and Development Co. Ltd., Rehovot (IL)

(21) Appl. No.: 17/736,190

(22) Filed: May 4, 2022

Related U.S. Application Data
(63) Continuation of application No. PCT/IL2020/051152, filed on Nov. 5, 2020.
(60) Provisional application No. 62/930,621, filed on Nov. 5, 2019.

(51) Int. Cl. A61K 35/28; A61K 35/17; A61P 35/00; C12N 5/0783

(52) U.S. Cl. CPC A61K 35/28; A61K 35/17; A61K 2039/5158; C12N 5/0636; A61P 35/00

(57) ABSTRACT

A method of treating or preventing a T cell mediated autoimmune disease in a subject in need thereof is disclosed. The method comprising: (a) transplanting immature hematopoietic cells into the subject; and (b) administering to the subject a therapeutically effective amount of an isolated population of non-GVHD inducing anti-third party cells comprising cells having a central memory T-lymphocyte (Tcm) phenotype, the cells being tolerance inducing cells and capable of homing to the lymph nodes following transplantation.

### RELATED APPLICATIONS

This application is a Continuation of PCT Patent Application No. PCT/IL2020/051152 having International filing date of Nov. 5, 2020, which claims the benefit of priority under 35 USC § 119(e) of U.S. Provisional Patent Application No. 62/930,621 filed on Nov. 5, 2019. The contents of the above applications are all incorporated by reference as if fully set forth herein in their entirety.

### SEQUENCE LISTING STATEMENT

The ASCII file, entitled 92305SequenceListing.txt, created on May 4, 2022, comprising 2,977 bytes, submitted concurrently with the filing of this application is incorporated herein by reference.

### FIELD AND BACKGROUND OF THE INVENTION

The present invention, in some embodiments thereof, relates to the use of tolerance inducing anti-third party cells comprising central memory T-lymphocyte phenotype as an adjuvant treatment for hematopoietic stem cell transplantation in treating T cell mediated autoimmune diseases.

Autoreactive T lymphocytes are key players in autoimmune diseases including autoimmune type 1 diabetes mellitus (T1DM), rheumatoid arthritis (RA), multiple sclerosis (MS) and celiac disease. In these diseases, failing immunological tolerance for critical self-antigens causes by the attack on various organs by the patient's own T cells. Despite the success of novel immunosuppressive biological drugs, none of these drugs leads to a permanent state of medicine free disease remission nor restores physiological mechanisms of self-tolerance.

For example, T1DM results from autoimmune attack on insulin-secreting beta cells and subsequent insulin deficiency. The cure of T1DM requires reversal of autoimmunity and resupply of insulin-secreting beta cells by islet transplantation or augmentation of endogenous beta cell regeneration.

The induction of tolerance towards pancreas autoantigens is a promising approach in the treatment of T1DM. This goal can be partially attained by immuno-ablation followed by autologous hematopoietic stem cell transplant (HSCT) which mitigates the immune reaction leading to diabetes, at least temporarily, if performed soon after diagnosis. However, most T1DM patients transplanted with autologous HSCT eventually relapse [Snarski E. et al., Bone marrow Transplantation (2016) 51, 398-402]. Thus, developing methodologies for allogeneic HSCT, to provide a durable non-autoimmune TCR repertoire, could be a promising treatment approach provided that such protocols can safely achieve donor type chimerism. Accordingly, transplantation of T cell-depleted allogeneic HSCT (TD-HSCT) under mild conditioning associated with minimal toxicity and reduced risk of GVHD, offers an attractive therapeutic option. However, overcoming graft rejection after reduced conditioning in T1DM represents a major challenge.

Reisner and co-workers previously demonstrated in wild type mice that rejection of TD-HSCT can be prevented using donor-derived veto cells. Veto activity, first defined by Miller [Miller, R. G, Nature (1980) 287; 544-54], is based on the ability of specific cell populations to attack host CTL-precursors (CTLp) that are directed against the antigens presented by the veto cells themselves. This response spares cells that are not targeted against the veto cells, including those recognizing pathogens. Among different veto cell populations described in the literature, central memory CD8 T cells exhibit the most robust veto activity upon transplantation, but are also endowed with marked graft-versus-host (GVH) activity. Reisner and co-workers overcame GVH reactivity by expanding naïve or memory CD8 T cells against 3rd party MHC or viral antigens, under culture conditions favoring expression of the central memory phenotype. Such anti-3rd party central memory CD8 T cells (Tcm) are endowed with marked veto activity, while effectively depleted of GVH reactivity in fully mis-matched recipients [Reviewed in Reisner Y, Or-Geva N. Semin. Hematol. (2019) 56(3): 173-182].

Various approaches have been contemplated for generation of tolerance inducing cells devoid of GVH reactivity and the use of same as an adjuvant treatment for graft transplantation, some are summarized infra.

PCT Publication No. WO 2001/049243 discloses non-alloreactive anti-third party cytotoxic T-lymphocytes (CTLs), wherein the non-alloreactive anti-third party CTLs are generated by directing T lymphocytes of the donor against 3^{rd}-party stimulators in the absence of exogenous IL-2. This approach was based on the observation that only activated cytotoxic T lymphocyte precursors (CTLp) were capable of surviving the IL-2 deprivation in the primary culture (IL-2 starvation results in apoptosis of non-induced T cells). Introduction of these anti-3^{rd} party veto CTLs into a recipient (along with a transplant) prevented graft rejection without inducing graft GVHD.

PCT Publication No. WO 2007/023491 discloses the use of tolerogenic cells for reducing or preventing graft rejection of a non-syngeneic graft in a subject. The tolerogenic T regulatory cells disclosed (e.g. CD4⁺CD25⁺ cells) may be derived from any donor who is non- syngeneic with both the subject and the graft (“third-party” tolerogenic cells). The graft (e.g. bone marrow) may be derived from any graft donor who is allogeneic or xenogeneic with the subject.

PCT Publication No. WO 2002/102971 discloses the use of cultured hematopoietic progenitor cells (HPC) comprising enhanced veto activity for inducing tolerance to a transplant transplanted from a donor to a recipient. The tolerogenic cells disclosed preferably express CD33 and are administered prior to, concomitantly with or following transplantation of the transplant (e.g. cell or organ transplant).

PCT Publication Nos. WO 2010/049935 and WO 2012/032526 disclose an isolated population of cells comprising non-GVHD inducing anti-third party cells having a Tcm phenotype, the cells being tolerance-inducing cells and capable of homing to the lymph nodes following transplantation. Specifically, WO 2010/049935 and WO 2012/032526 teach co-transplantation of immature hematopoietic stem cells along with the anti-third party Tcm cells. The use of the anti-third party Tcm cells enabled engraftment of immature hematopoietic cells without graft versus host disease (GVHD).

PCT Publication Nos. WO 2013/035099 and WO 2018/002924 disclose methods of generating an isolated population of cells comprising anti-third party cells having a Tcm phenotype, the cells being tolerance-inducing cells and/or endowed with anti-disease activity, and capable of homing to the lymph nodes following transplantation.

### SUMMARY OF THE INVENTION

According to an aspect of some embodiments of the present invention there is provided a method of treating or preventing a T cell mediated autoimmune disease in a subject in need thereof, the method comprising: (a) transplanting immature hematopoietic cells into the subject; and (b) administering to the subject a therapeutically effective amount of an isolated population of non-GVHD inducing anti-third party cells comprising cells having a central memory T-lymphocyte (Tcm) phenotype, the cells being tolerance inducing cells and capable of homing to the lymph nodes following transplantation, thereby treating the T cell mediated autoimmune disease in the subject.

According to an aspect of some embodiments of the present invention there is provided an immature hematopoietic cell transplant and a therapeutically effective amount of an isolated population of non-GVHD inducing anti-third party cells comprising cells having a central memory T-lymphocyte (Tcm) phenotype, the cells being tolerance inducing cells and capable of homing to the lymph nodes following transplantation, for use in treating or preventing a T cell mediated autoimmune disease in a subject in need thereof.

According to some embodiments of the invention, the isolated population of non-GVHD inducing anti-third party cells are co-administrated with the immature hematopoietic cell transplant.

According to some embodiments of the invention, the isolated population of non-GVHD inducing anti-third party cells are for administration following the immature hematopoietic cell transplant.

According to some embodiments of the invention, the isolated population of non-GVHD inducing anti-third party cells are for administration on day 1-20 following the immature hematopoietic cell transplant.

According to some embodiments of the invention, the isolated population of non-GVHD inducing anti-third party cells are for administration at a dose of at least 0.5×10⁶ CD8⁺ cells per kg ideal body weight.

According to some embodiments of the invention, the isolated population of non-GVHD inducing anti-third party cells are for administration at a dose of 5×10⁶-10×10⁶ CD8⁺ cells per kg ideal body weight.

According to some embodiments of the invention, the isolated population of non-GVHD inducing anti-third party cells are used for reducing graft rejection and/or inducing donor specific tolerance.

According to some embodiments of the invention, the isolated population of non-GVHD inducing anti-third party cells are used as an adjuvant treatment for reducing graft rejection and/or inducing donor specific tolerance.

According to some embodiments of the invention, the isolated population of non-GVHD inducing anti-third party cells are generated by a method comprising:

(a) contacting peripheral blood mononuclear cells (PBMCs) with a third party antigen or antigens in a culture deprived of cytokines so as to allow enrichment of antigen reactive cells; and

(b) culturing the cells resulting from step (a) in the presence of cytokines so as to allow proliferation of cells comprising the central memory T-lymphocyte (Tcm) phenotype, thereby generating the non-GVHD inducing anti-third party cells.

According to some embodiments of the invention, the method further comprises depleting CD4+ and/or CD56+ expressing cells from the PBMCs prior to the contacting with the third party antigen or antigens.

According to some embodiments of the invention, the method further comprises selecting CD45RA⁺ expressing cells so as to obtain a population of naïve T cells expressing a CD45RA⁺CD8⁺ phenotype.

According to some embodiments of the invention, the method further comprises depleting CD45RA⁺ expressing cells so as to obtain a population enriched of memory T cells expressing a CD45RA⁻CD8⁺ phenotype.

According to some embodiments of the invention, the contacting with the antigen or antigens of step (a) in effected in the presence of IL-21.

According to some embodiments of the invention, the culturing the cells resulting from step (a) in the presence of cytokines comprises culturing the cells in the presence of IL-15.

According to some embodiments of the invention, the culturing the cells resulting from step (a) in the presence of cytokines comprises culturing the cells in the presence of IL-21, IL-15 and/or IL-7.

According to some embodiments of the invention, the antigen or antigens is selected from the group consisting of a viral antigen, a bacterial antigen, a tumor antigen, an autoimmune disease related antigen, a protein extract, a purified protein and a synthetic peptide.

According to some embodiments of the invention, the antigen or antigens is presented by autologous antigen presenting cells, non-autologous antigen presenting cells, artificial vehicles or artificial antigen presenting cells.

According to some embodiments of the invention, the antigen or antigens is presented by antigen presenting cells of the same origin as the PBMCs.

According to some embodiments of the invention, the antigen or antigens comprises stimulatory cells selected from the group consisting of cells purified from peripheral blood lymphocytes, spleen or lymph nodes, cytokine-mobilized PBLs, in vitro expanded antigen-presenting cells (APC), in vitro expanded dendritic cells and artificial antigen presenting cells.

According to some embodiments of the invention, the method further comprises selecting for CD3⁺, CD8⁺, CD62L⁺, CD45RA⁻, CD45RO⁺ signature.

According to some embodiments of the invention, the immature hematopoietic cells comprise T cell depleted immature hematopoietic cells.

According to some embodiments of the invention, the immature hematopoietic cells comprise at least 5×10⁶ CD34⁺ cells per kilogram ideal body weight of the subject.

According to some embodiments of the invention, the immature hematopoietic cells are depleted of CD3⁺ and/or CD19⁺ expressing cells.

According to some embodiments of the invention, the immature hematopoietic cells comprise less than 5×10⁵ CD3⁺ expressing cells per kg ideal body weight of the subject.

According to some embodiments of the invention, the immature hematopoietic cell transplant is non-syngeneic with the subject.

According to some embodiments of the invention, the immature hematopoietic cell transplant and the isolated population of non-GVHD inducing anti-third party cells are obtained from the same donor.

According to some embodiments of the invention, the method further comprises conditioning the subject under non-myeloablative conditioning (e.g. prior to the transplanting).

According to some embodiments of the invention, the method further comprises a non-myeloablative conditioning (e.g. pre-transplant conditioning).

According to some embodiments of the invention, the non-myeloablative conditioning comprises at least one of total body irradiation (TBI), a partial body irradiation (TLI), a chemotherapeutic agent, an antibody immunotherapy or a co-stimulatory blockade.

According to some embodiments of the invention, the TBI comprises an irradiation dose (e.g. single or fractionated irradiation dose) within the range of 1-6 Gy.

According to some embodiments of the invention, the TBI is to be administered on any one of days −3 to 0 of transplanting.

According to some embodiments of the invention, the TBI is to be administered on any one of days −3 to −1 prior to the transplanting.

According to some embodiments of the invention, the TBI is to be administered one or two days prior to the transplanting.

According to some embodiments of the invention, the chemotherapeutic agent comprises at least one of Everolimus, Fludarabine, Cyclophosphamide, Busulfan, Trisulphan, Melphalan or Thiotepa.

According to some embodiments of the invention, the method further comprises administering to the subject a therapeutically effective amount of Rapamycin.

According to some embodiments of the invention, the method further comprises a therapeutically effective amount of Rapamycin.

According to some embodiments of the invention, the therapeutically effective amount of Rapamycin comprises at least 0.1 mg Rapamycin per day per kilogram ideal body weight of the subject.

According to some embodiments of the invention, the Rapamycin is to be administered to the subject on days −1 to +4 of the transplanting.

According to some embodiments of the invention, the non-myeloablative conditioning comprises T cell debulking.

According to some embodiments of the invention, the T cell debulking is effected by at least one of anti-thymocyte globulin (ATG) antibodies, anti-CD52 antibodies or anti-CD3 (OKT3) antibodies.

According to some embodiments of the invention, the non-myeloablative conditioning comprises a therapeutically effective amount of Fludarabine.

According to some embodiments of the invention, the method further comprises administering to the subject a therapeutically effective amount of cyclophosphamide.

According to some embodiments of the invention, the method further comprises a therapeutically effective amount of cyclophosphamide.

According to some embodiments of the invention, the therapeutically effective amount of cyclophosphamide comprises 25-200 mg per kilogram ideal body weight of the subject.

According to some embodiments of the invention, the cyclophosphamide is to be administered to the subject on days +3 and +4 of the transplanting.

According to some embodiments of the invention, the method comprises:

(a) conditioning the subject under non-myeloablative conditioning, wherein the non-myeloablative conditioning comprises a total body irradiation (TBI) and a immunosuppressive agent, wherein the TBI and the immunosuppressive agent are administered on days −4 to +4 of transplantation;

(b) transplanting into the subject a dose of T cell depleted immature hematopoietic cells, wherein the T cell depleted immature hematopoietic cells comprises at least 5×10⁶ CD34⁺ cells per kilogram ideal body weight of the subject; and

(c) administering to the subject a therapeutically effective amount of an isolated population of non-GVHD inducing anti-third party cells comprising cells having a central memory T-lymphocyte (Tcm) phenotype, the cells being tolerance inducing cells and capable of homing to the lymph nodes following transplantation.

According to some embodiments of the invention, the immunosuppressive agent comprises Rapamycin.

According to some embodiments of the invention, the Rapamycin is administered on days −1 to +4 of the transplantation.

According to some embodiments of the invention, the TBI is administered on days −3 to 0 of transplantation.

According to some embodiments of the invention, the TBI is administered on days −1 prior to transplantation.

According to some embodiments of the invention, step (b) and step (c) are effected concomitantly.

According to some embodiments of the invention, the isolated population of non-GVHD inducing cells is administered on day 1-20 following the transplantation of the T cell depleted immature hematopoietic cells.

According to some embodiments of the invention, the method comprises:

(a) conditioning the subject under non-myeloablative conditioning, wherein the non-myeloablative conditioning comprises a total body irradiation (TBI) and a chemotherapeutic agent, wherein the TBI and the chemotherapeutic agent are administered on days −6 to 0 of transplantation;

(b) transplanting into the subject a dose of T cell depleted immature hematopoietic cells, wherein the T cell depleted immature hematopoietic cells comprises at least 5×10⁶ CD34⁺ cells per kilogram ideal body weight of the subject;

(c) administering to the subject a therapeutically effective amount of cyclophosphamide, wherein the therapeutically effective amount of the cyclophosphamide comprises 25-200 mg cyclophosphamide per kilogram ideal body weight of the subject, and wherein the therapeutically effective amount of the cyclophosphamide is to be administered to the subject in two doses on days +3 and +4 following the transplantation of the T cell depleted immature hematopoietic cells; and

(d) administering to the subject a therapeutically effective amount of an isolated population of non-GVHD inducing anti-third party cells comprising cells having a central memory T-lymphocyte (Tcm) phenotype, the cells being tolerance inducing cells and capable of homing to the lymph nodes following transplantation, wherein the isolated population of non-GVHD inducing cells are administered on day +5 to +10 following the transplantation of the T cell depleted immature hematopoietic cells.

According to some embodiments of the invention, the chemotherapeutic agent comprises Fludarabine.

According to some embodiments of the invention, the Fludarabine is administered on days −6 to −3 prior to the transplantation.

According to some embodiments of the invention, the TBI is administered on day −1 prior to the transplantation.

According to some embodiments of the invention, the method further comprises T cell debulking prior to step (a).

According to some embodiments of the invention, the T cell debulking is effected by anti-thymocyte globulin (ATG) administered on days −9 to −7 prior to the transplantation.

According to some embodiments of the invention, the isolated population of non-GVHD inducing cells is administered on day +7 following the transplantation of T cell depleted immature hematopoietic cells.

According to some embodiments of the invention, the T cell mediated autoimmune disease is selected from the group consisting of type 1 diabetes mellitus (T1DM), Hashimoto's thyroiditis, multiple sclerosis (MS), rheumatoid arthritis (RA), ankylosing spondylitis, Crohn's disease, ulcerative colitis (UC), non-infectious uveitis, Lupus erythematosus (SLE), Sjogren's syndrome, primary biliary cirrhosis, autoimmune hepatitis, Immune Thrombocytic Purpura (ITP), Chronic Glomerulonephritis, Myasthenia gravis, Systemic Scleroderma, Polymyositis and Addison's disease.

According to some embodiments of the invention, when the T cell mediated autoimmune disease is type 1 diabetes mellitus (T1DM), the transplant further comprises a pancreatic cell or tissue transplant.

According to some embodiments of the invention, the pancreatic cell or tissue transplant is from the same donor as the immature hematopoietic cell transplant and/or the isolated population of non-GVHD inducing anti-third party cells.

According to some embodiments of the invention, when the T cell mediated autoimmune disease is a primary biliary cirrhosis or an autoimmune hepatitis, the transplant further comprises a hepatic cell or tissue transplant.

According to some embodiments of the invention, the hepatic cell or tissue transplant is from the same donor as the immature hematopoietic cell transplant and/or the isolated population of non-GVHD inducing anti-third party cells.

According to some embodiments of the invention, the subject is a human subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of a reduced intensity conditioning (RIC) protocol of T cell depleted bone marrow transplant.

FIGS. 2A-B illustrate diabetes free survival and transplant-related mortality following MHC disparate non-myeloablative bone marrow transplant (BMT) in NOD mice. Transplantation of megadose NuBM (H-2^{b}) cells into NOD (H-2^{d}) recipient mice with 5×10⁶ Tcm on day 0. Hosts were conditioned with 4.5 Gy TBI and treated with Rapamycin (12.5 mg/kg) on days −1 to +4. Control groups received no treatment, or were subjected to conditioning but without a transplant. (FIG. 2A) Kaplan Meyer curve presenting diabetes-free survival. (FIG. 2B) Kaplan Meyer curve presenting non-diabetes related mortality.

FIGS. 3A-J illustrate that reduced intensity bone marrow transplantation in NOD mice enables engraftment and prevents development of diabetes. Donor type chimerism 6 months post transplantation of megadose C57BL/6 nude (H-2^{b}) BM cells into NOD mice (H-2^{d}) recipient mice with 5×10⁶ Tcm on day 0. Hosts were conditioned with 4.5 Gy TBI and injected with Rapamycin (12.5 μg/mouse) at days −1 to +4. Control groups did not receive conditioning or BM. (FIG. 3A) Percentage of donor cells in peripheral blood was analyzed by FACS Scatter plots using anti-donor and anti-host (H-2^{d} and H-2^{b}) antibodies; each dot represents one mouse belonging to the appropriate group; (FIGS. 3B-F) Flow cytometry analysis of CD4 T cells (CD45⁺CD3⁺CD4⁺), CD8 T cells (CD45⁺CD3⁺CD8⁺) and B cells (CD45⁺CD3⁻CD19⁺) from spleen and blood of treatment group (4.5 Gy+Rapa+BM+Tcm) and no treatment mice; (FIGS. 3G-J) Flow cytometry analysis of DCs (CD45⁺CD11b⁺MHC-II⁺) and Macrophages (CD45⁺CD11b⁺F4/80⁺) from spleen of treatment group (4.5 Gy+Rapa+BM+Tcm) and no treatment mice. (N≥3).

FIGS. 4A-B illustrate Diabetes free survival and transplant-related mortality following mismatch MHC non-myeloablative BMT in NOD mice. Transplantation of megadose C57BL/6 nude (H-2^{b}) cells into NOD (H-2^{d}) recipient mice with 5×10⁶ Tcm on day 0. Hosts were conditioned with 4.5 Gy TBI and treated with Rapamycin (12.5 μg/mouse) on days −1 to +4. Control groups received no treatment or were subjected to conditioning but without a transplant. (FIG. 4A) Kaplan Meyer curve presenting diabetes-free survival; (FIG. 4B) Kaplan Meyer curve presenting non-diabetes related mortality.

FIG. 5 is a table illustrating diabetes free survival, transplant-related mortality and disease mortality following mismatch MHC non-myeloablative BMT in NOD mice. Presented are detailed data from 4 independent experiments.

FIGS. 6A-D are photographs illustrating that donor-type CD8⁺ veto cells survive in the host's lymphoid organs a year post transplantation. Presence of GFP-marked Tcm cells one-year post transplantation of megadose C57BL/6 nude (H-2^{b}) BM cells into NOD mice (H-2^{d}) recipient mice with 5×10⁶ Tcm-GPF on day 0. Hosts were conditioned with 4.5 Gy TBI and injected with Rapamycin (12.5 μg/mouse) at days −1 to +4. Lymphoid organs of 3 different mice were evaluated. Imaging Acquisition by Two-photon laser scanning microscope (Zeis). (FIG. 6A) popliteal lymph node; (FIG. 6B) Zoom in-popliteal lymph node; (FIG. 6C) mesenteric lymph node; and (FIG. 6D) spleen.

FIGS. 7A-B illustrate T cells repertoire analysis. (FIG. 7A) Venn plot demonstrating the number of CDR3 amino acids in the b-chain sequences shared across different mice groups. Of note, treated mice share less amino acids in the beta-chain sequence; (FIG. 7B) V beta usage of T cell types—PC analysis. PC1 separates between CD4, CD8 and CD25 cells. PC2 divides between clones from treated and non-treated mice.

FIG. 8 is a table illustrating that autoimmune TCRs are not found in NOD mice post transplantation. CDR3 beta amino chains related to autoimmunity were demonstrated in NOD mice before and after development of diabetes but were not found in NOD mice post transplantation.

### DESCRIPTION OF SPECIFIC EMBODIMENTS OF THE INVENTION

The present invention, in some embodiments thereof, relates to the use of tolerance inducing anti-third party cells comprising central memory T-lymphocyte phenotype as an adjuvant treatment for hematopoietic stem cell transplantation in treating T cell mediated autoimmune diseases.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Autoreactive T lymphocytes are key players in autoimmune diseases including autoimmune type 1 diabetes mellitus (T1DM), rheumatoid arthritis (RA), multiple sclerosis (MS), systemic lupus erythematosus (SLE) and celiac disease. In these diseases, failing immunological tolerance for critical self-antigens causes by the attack on various organs by the patient's own T cells. Allogeneic hematopoietic stem cell transplant (HSCT), which can potentially “reset” the immune system in autoimmunity and may provide a durable non-autoimmune TCR repertoire, can be a promising treatment approach provided that such protocols can safely achieve donor type chimerism with minimal toxicity and reduced risk of graft rejection and GVHD.

While reducing the present invention to practice, the present inventors have uncovered that donor-derived veto cells generated by third party stimulation can be used safely and efficiently for prevention of graft rejection and GVHD of HSCT in the treatment of T cell mediated autoimmune diseases including T1DM.

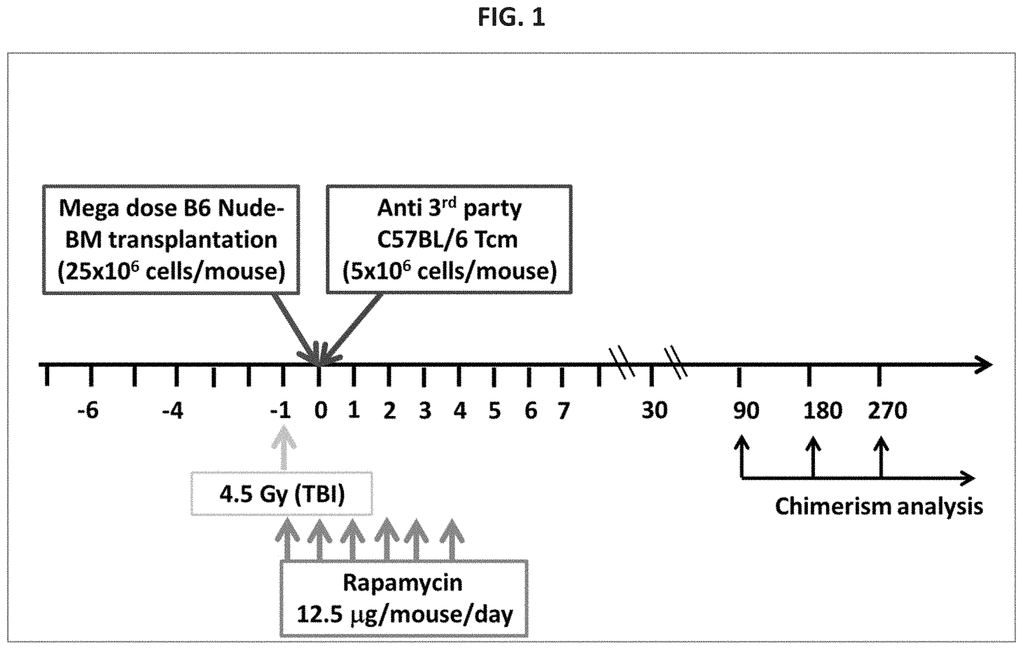

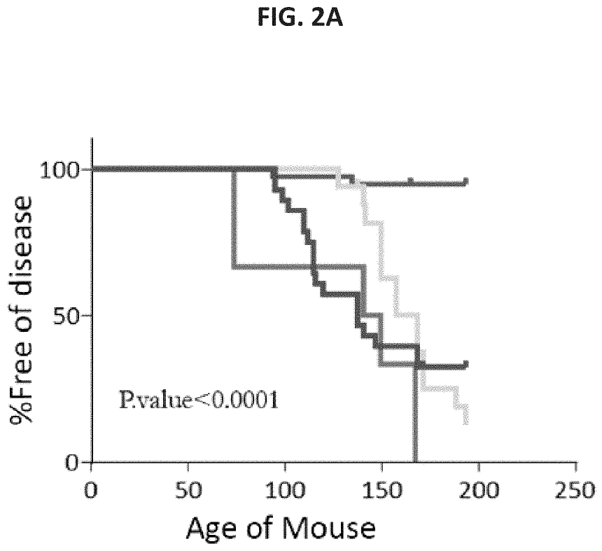

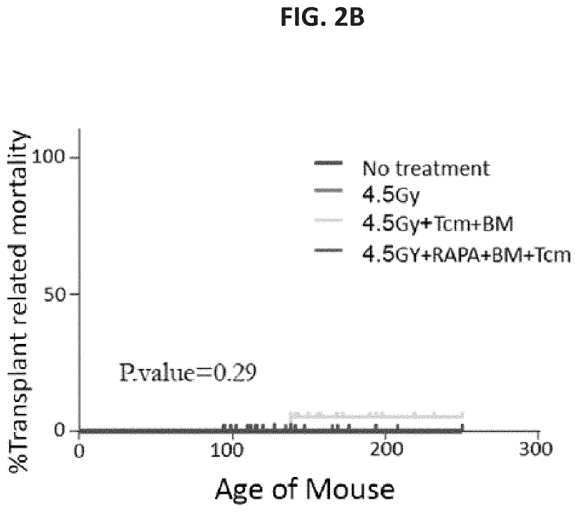

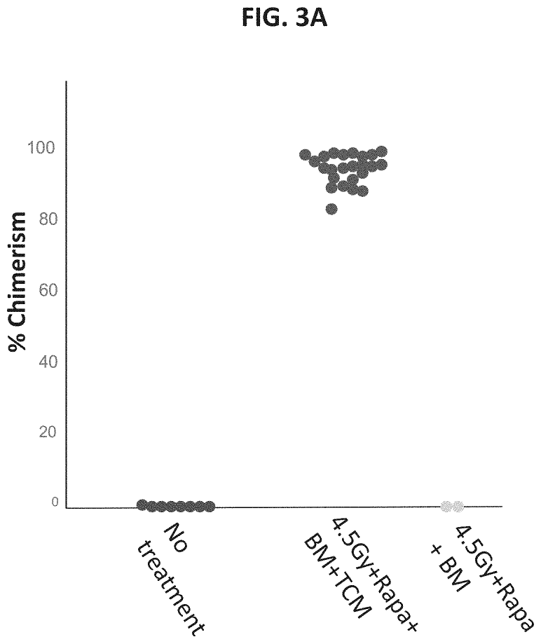

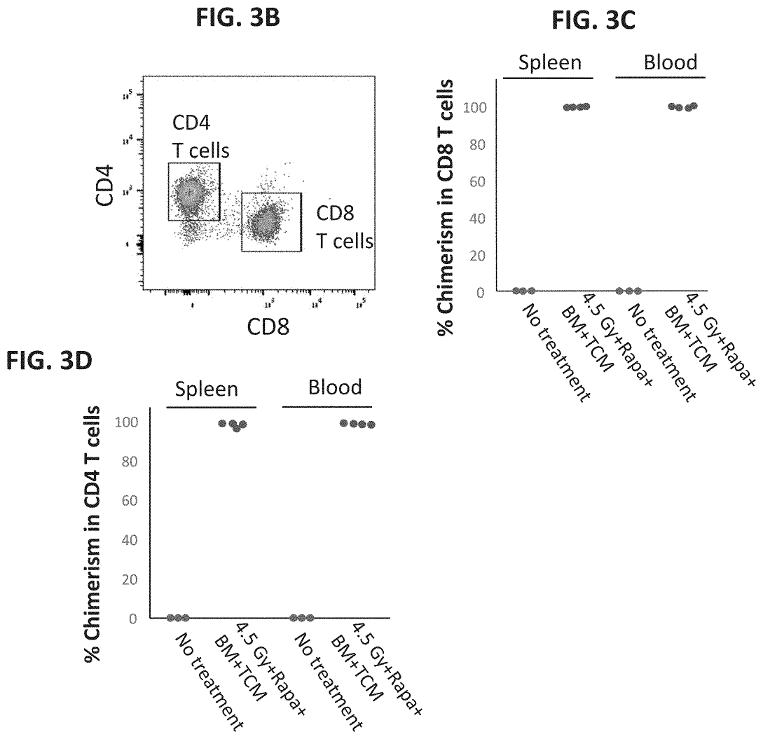

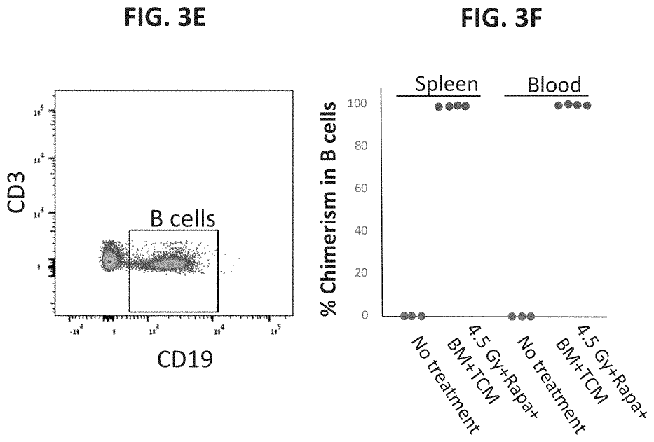

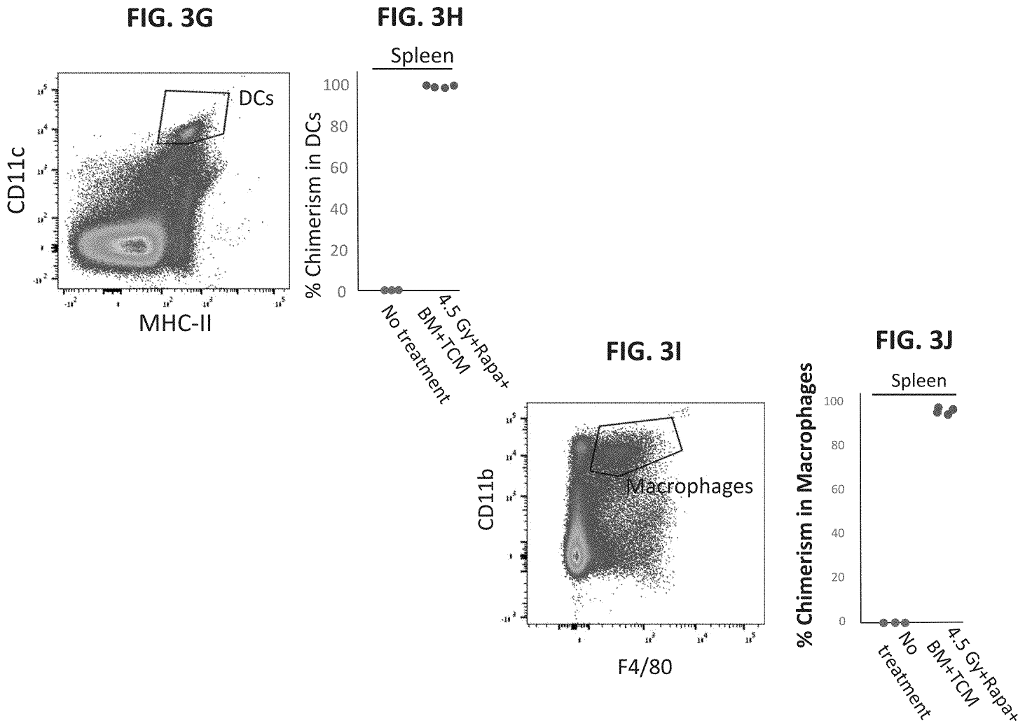

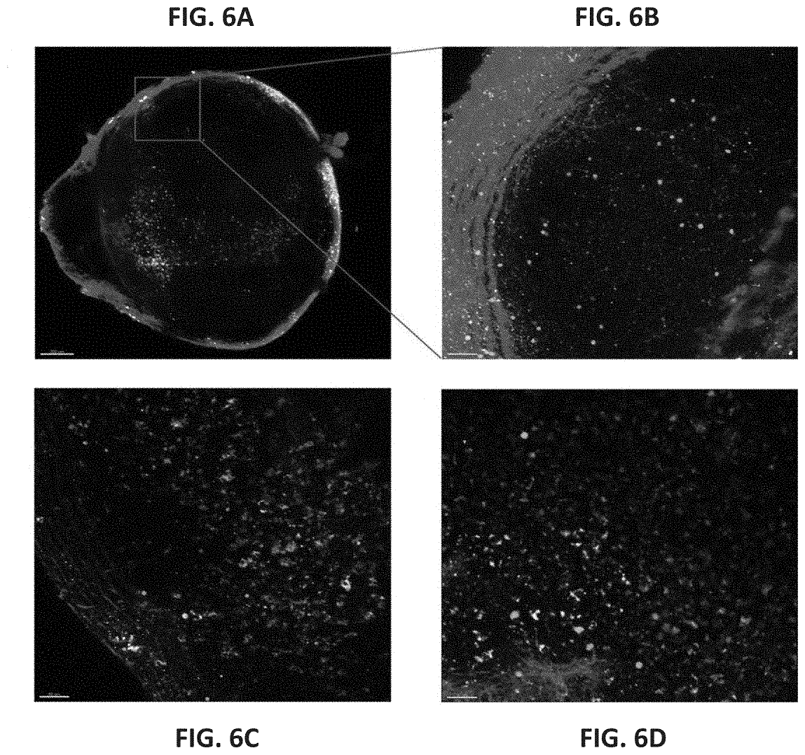

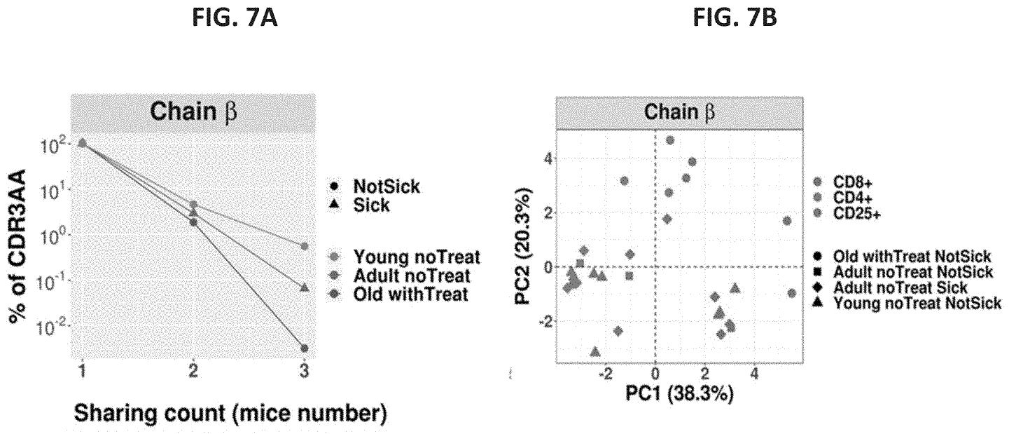

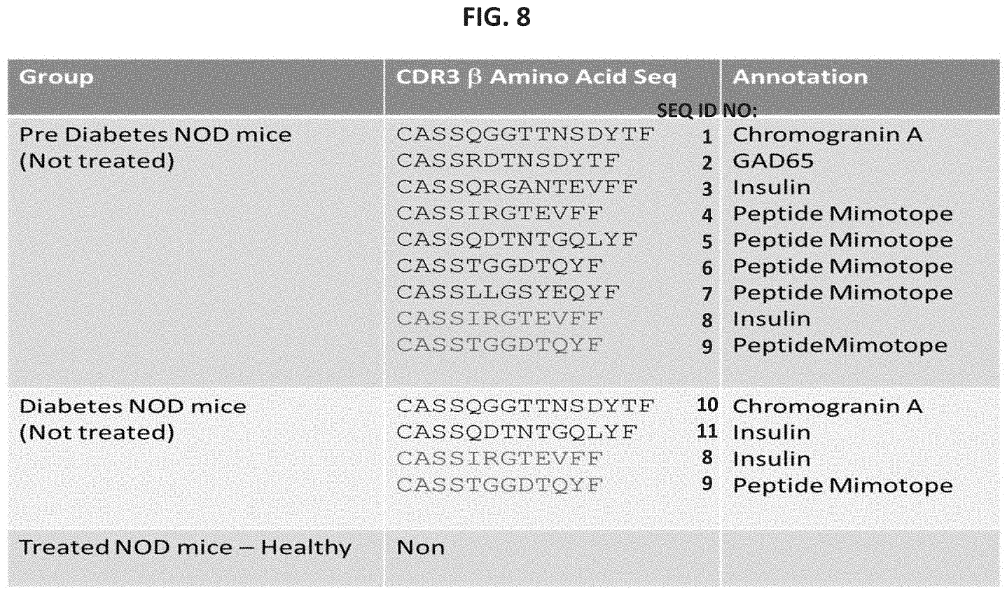

As is shown herein below and in the Examples section which follows, the present inventors have uncovered through laborious experimentation a low toxicity protocol for treatment of autoimmune type 1 diabetes mellitus comprising transplantation of T cell depleted MHC disparate bone marrow cells and anti-third party veto Tcm cells (see FIG. 1). According to the treatment protocol, the subject is first treated by a reduced intensity conditioning including total body irradiation (TBI) on day −1 and short term Rapamycin on days −1 to +4, followed by T cell depleted bone marrow transplant (on day 0) along with anti-third party veto Tcm cells (on day 0). This protocol enabled long term diabetes free survival in the absence of graft rejection and GVHD in the engrafted mice (see FIGS. 2A-B, 3A and 4A-B). Although minimal conditioning was used, chimerism analysis in the transplanted mice showed multi-lineage chimerism, in various cell types of the immune system, including myeloid cells, dendritic cells and lymphoid cells, implying the extended change in the hosts' immune system (see FIGS. 3B-J). Notable, analysis of lymphoid organs of treated NOD mice, using GFP-allogeneic Tcm, showed survival of Tcm in spleen and various lymph nodes up to one-year post transplant (see FIGS. 6A-D), illustrating that durable immune tolerance was obtained. Moreover, TCR sequencing analysis showed that sequences related to diabetes autoimmunity, typical of autoimmune T cell clones, were not found in the treated mice (see FIGS. 7A-B), suggesting that a deletion-based mechanism is underlying the prevention of disease onset in the transplanted mice. Sequencing analysis also revealed different CD8 and CD4 vβ usage in treated mice versus not treat mice (see FIG. 8). These results illustrate that although BM pluripotent cells were “educated” in the same thymus, different T cells were generated and autoimmunity was prevented upon bone marrow transplantation.

Taken together, the anti-third party veto Tcm cells offer treatment of T cell mediated autoimmune diseases including T1DM by enabling transplantation of MHC disparate HSCT following a short course of reduced intensity conditioning in the absence of GVHD and graft rejection.

Thus, according to one aspect of the present invention there is provided a method of treating or preventing a T cell mediated autoimmune disease in a subject in need thereof, the method comprising: (a) transplanting immature hematopoietic cells into the subject; and (b) administering to the subject a therapeutically effective amount of an isolated population of non-GVHD inducing anti-third party cells comprising cells having a central memory T-lymphocyte (Tcm) phenotype, the cells being tolerance inducing cells and capable of homing to the lymph nodes following transplantation.

According to another aspect of the present invention there is provided an immature hematopoietic cell transplant and a therapeutically effective amount of an isolated population of non-GVHD inducing anti-third party cells comprising cells having a central memory T-lymphocyte (Tcm) phenotype, the cells being tolerance inducing cells and capable of homing to the lymph nodes following transplantation, for use in treating or preventing a T cell mediated autoimmune disease in a subject in need thereof.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the term “preventing” refers to keeping a disease, disorder or condition from occurring in a subject who may be at risk for the disease or disorder, but has not yet been diagnosed as having the disease or disorder, e.g. a T cell mediated autoimmune disease.

As used herein, the term “subject” or “subject in need thereof” refers to a mammal, preferably a human being, male or female, at any age that suffers from a T cell mediated autoimmune disease or is at high risk of developing a T cell mediated autoimmune disease (e.g. has genetic predisposition for a T cell mediated autoimmune disease). Typically the subject is in need of an immature hematopoietic cell transplantation (also referred to herein as recipient) for the treatment or prevention of the T cell mediated autoimmune disease.

As used herein, the term “T cell mediated autoimmune disease” refers to a disease or disorder in which an immune response involving T lymphocytes is generated in response to a substance, such as a protein or a tissue, that is normally present in the body and such response is undesirable (e.g. to one or more self-antigens).

Autoimmune diseases may also include diseases induced by foreign antigens, such as celiac disease.

Non-limiting examples of autoimmune diseases include, but are not limited to, Acute disseminated encephalomyelitis (ADEM), Addison's disease, Agammaglobulinemia, Alopecia areata, Amyotrophic lateral sclerosis, Ankylosing Spondylitis, Antiphospholipid syndrome, Antisynthetase syndrome, Atopic allergy, Atopic dermatitis, Autoimmune aplastic anemia, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenic purpura, Autoimmune uveitis (e.g. non-infectious uveitis), Behcet's disease, Celiac disease, Chronic Glomerulonephritis, Cold agglutinin disease, Crohn's disease, Dermatomyositis, Dermatomyositis, Diabetes mellitus type 1 (Type 1 diabetes mellitus (T1DM)), Eosinophilic fasciitis, Goodpasture's syndrome, Graves' disease, Guillain-Barre syndrome (GBS), Hashimoto's encephalopathy, Hashimoto's thyroiditis, Idiopathic thrombocytopenic purpura, Immune Thrombocytic Purpura (ITP), Lupus erythematosus (SLE), Miller-Fisher syndrome, Mixed connective tissue disease, Multiple sclerosis (MS), Myasthenia gravis, Narcolepsy, Pemphigus vulgaris, Pernicious anaemia, Polymyositis Primary biliary cirrhosis, Psoriasis, Psoriatic arthritis, Relapsing polychondritis, Rheumatoid arthritis (RA), Rheumatic fever, Systemic Scleroderma, Sjogren's syndrome, Temporal arteritis, Transverse myelitis, Ulcerative colitis (UC), Undifferentiated connective tissue disease, Vasculitis, and Wegener's granulomatosis.

According to a specific embodiment, the T cell mediated autoimmune disease is autoimmune type 1 diabetes mellitus (T1DM).

Type 1 diabetes mellitus (T1DM) is the result of organ-specific autoimmune destruction of insulin-secreting β-cells in the pancreatic islets of Langerhans, which eventually leads to no production of insulin by the body. Without enough insulin, glucose builds up in the bloodstream rather than being taken up by cells and the body is unable to use this glucose for energy.

Type 1 diabetes can occur at any age, however, it is often diagnosed in children, adolescents, or young adults. β-cell destruction starts well before the appearance of disease symptoms. It has been estimated that only 10% of the total β-cell mass remains at the time of clinical onset. Therefore, when the disease is diagnosed, critical β-cell depletion and insulin dependency may have already occurred.

Symptoms of T1DM include, without being limited to, high levels of thirst and hunger, frequent urination, fatigue, blurry eyesight, numbness or tingling in the feet, unintentional weight loss, deep and rapid breathing, dry skin and mouth, flushed face, fruity breath odor, nausea or vomiting, inability to keep down fluids, and stomach pain.

Treatment of T cell mediated autoimmune disease is affected by transplantation of immature hematopoietic cells into the subject.

As used herein, the phrase “transplantation” refers to administration of a bodily cell, e.g. a single cell or a group of cells, into a subject.

As used herein the phrase “immature hematopoietic cells” refers to a hematopoietic tissue or cell preparation comprising precursor hematopoietic cells (e.g. hematopoietic stem cells). Such tissue/cell preparation includes or is derived from a biological sample, for example, bone marrow, mobilized peripheral blood (e.g. mobilized CD34⁺ expressing cells to enhance their concentration), cord blood (e.g. umbilical cord), fetal liver, yolk sac and/or placenta. Additionally, purified CD34⁺ cells or other hematopoietic stem cells such as CD131⁺ cells can be used in accordance with the present teachings, either with or without ex-vivo expansion.

According to one embodiment, the immature hematopoietic cells comprise T cell depleted immature hematopoietic cells.

As used herein the phrase “T cell depleted immature hematopoietic cells” refers to a population of precursor hematopoietic cells which are depleted of T lymphocytes. The T cell depleted immature hematopoietic cells, may include e.g. CD34⁺, CD33⁺ and/or CD56⁺ cells. The T cell depleted immature hematopoietic cells may be depleted of CD3⁺ cells, CD2⁺ cells, CD8⁺ cells, CD4⁺ cells, α/β T cells, and/or γ/δ T cells.

According to one embodiment, the immature hematopoietic cells comprise T cell depleted mobilized blood cells enriched for CD34⁺ immature hematopoietic cells.

According to one embodiment, the T cell depleted immature hematopoietic cells comprise 0.1×10⁶-20×10⁶ CD34⁺ cells (e.g. 1×10⁶-10×10⁶ CD34⁺ cells) per kg ideal body weight of the subject.

According to an embodiment, the T cell depleted immature hematopoietic cells comprise at least about 0.1×10⁶ CD34⁺ cells, 0.5×10⁶ CD34⁺ cells, 1×10⁶ CD34⁺ cells, 2×10⁶ CD34⁺ cells, 3×10⁶ CD34⁺ cells, 4×10⁶ CD34⁺ cells, 5×10⁶ CD34⁺ cells, 6×10⁶ CD34⁺ cells, 7×10⁶ CD34⁺ cells, 8×10⁶ CD34⁺ cells, 9×10⁶ CD34⁺ cells, 10×10⁶ CD34⁺ cells, 15×10⁶ CD34⁺ cells or 20×10⁶ CD34⁺ cells per kg ideal body weight of the subject.

According to a specific embodiment, the T cell depleted immature hematopoietic cells comprise at least about 5×10⁶ CD34⁺ cells per kg ideal body weight of the subject.

According to a specific embodiment, the T cell depleted immature hematopoietic cells comprise at least about 6×10⁶ CD34⁺ cells per kg ideal body weight of the subject.

According to a specific embodiment, the T cell depleted immature hematopoietic cells comprise at least about 8×10⁶ CD34⁺ cells per kg ideal body weight of the subject.

According to a specific embodiment, the T cell depleted immature hematopoietic cells comprise at least about 10×10⁶ CD34⁺ cells per kg ideal body weight of the subject.

According to one embodiment, the immature hematopoietic cells are depleted of CD3⁺ and/or CD19⁺ cells.

According to an embodiment, the T cell depleted immature hematopoietic cells comprise less than 1×10⁴-1×10⁶ (e.g. 1×10⁵-50×10⁵) CD3⁺ cells per kilogram ideal body weight of the subject.

According to an embodiment, the T cell depleted immature hematopoietic cells comprise less than about 50×10⁵ CD3⁺ cells, 40×10⁵ CD3⁺ cells, 30×10⁵ CD3⁺ cells, 20×10⁵ CD3⁺ cells, 15×10⁵ CD3⁺ cells, 10×10⁵ CD3⁺ cells, 9×10⁵ CD3⁺ cells, 8×10⁵ CD3⁺ cells, 7×10⁵ CD3⁺ cells, 6×10⁵ CD3⁺ cells, 5×10⁵ CD3⁺ cells, 4×10⁵ CD3⁺ cells, 3×10⁵ CD3⁺ cells, 2×10⁵ CD3⁺ cells, 1×10⁵ CD3⁺, 0.5×10⁵ CD3⁺ or 0.1×10⁵ CD3⁺ cells per kilogram ideal body weight of the subject.

According to a specific embodiment, the T cell depleted immature hematopoietic cells comprise less than 5×10⁵ CD3⁺ cells per kilogram ideal body weight of the subject.

According to a specific embodiment, the T cell depleted immature hematopoietic cells comprise less than 3×10⁵ CD3⁺ cells per kilogram ideal body weight of the subject.

According to a specific embodiment, the T cell depleted immature hematopoietic cells comprise less than 2×10⁵ CD3⁺ cells per kilogram ideal body weight of the subject.

According to a specific embodiment, the T cell depleted immature hematopoietic cells comprise less than 1×10⁵ CD3⁺ cells per kilogram ideal body weight of the subject.

According to one embodiment, the immature hematopoietic cells are depleted of CD8⁺ cells.

According to an embodiment, the T cell depleted immature hematopoietic cells comprise less than 1×10⁴-1×10⁶ CD8⁺ cells (e.g. 1×10⁴-4×10⁵ CD8⁺ cells) per kilogram ideal body weight of the subject.

According to an embodiment, the T cell depleted immature hematopoietic cells comprise less than about 50×10⁵ CD8⁺ cells, 25×10⁵ CD8⁺ cells, 15×10⁵ CD8⁺ cells, 10×10⁵ CD8⁺ cells, 9×10⁵ CD8⁺ cells, 8×10⁵ CD8⁺ cells, 7×10⁵ CD8⁺ cells, 6×10⁵ CD8⁺ cells, 5×10⁵ CD8⁺ cells, 4×10⁵ CD8⁺ cells, 3×10⁵ CD8⁺ cells, 2×10⁵ CD8⁺ cells, 1×10⁵ CD8⁺ cells, 9×10⁴ CD8⁺ cells, 8×10⁴ CD8⁺ cells, 7×10⁴ CD8⁺ cells, 6×10⁴ CD8⁺ cells, 5×10⁴ CD8⁺ cells, 4×10⁴ CD8⁺ cells, 3×10⁴ CD8⁺ cells, 2×10⁴ CD8⁺ cells or 1×10⁴ CD8⁺ cells per kilogram ideal body weight of the subject.

According to a specific embodiment, the T cell depleted immature hematopoietic cells comprise less than 4×10⁵ CD8⁺ cells per ideal kilogram body weight of the subject.

According to a specific embodiment, the T cell depleted immature hematopoietic cells comprise less than 1×10³-1×10⁶ CD8⁺ TCRα/β⁻ cells (e.g. 1×10⁴-1×10⁵ CD8⁺ TCRα/β⁻ cells) per kilogram ideal body weight of the subject.

According to an embodiment, the T cell depleted immature hematopoietic cells comprise less than about 1×10⁶ CD8⁺ TCRα/β⁻ cells, 0.5×10⁶ CD8⁺ TCRα/β⁻ cells, 1×10⁵ CD8⁺ TCRα/β⁻ cells, 0.5×10⁵ CD8⁺ TCRα/β⁻ cells, 1×10⁴ CD8⁺ TCRα/β⁻ cells, 0.5×10⁴ CD8⁺ TCRα/β⁻ cells, 1×10³ CD8⁺ TCRα/β⁻ cells or 0.5×10³ CD8⁺ TCRα/β⁻ cells per kilogram ideal body weight of the subject.

According to a specific embodiment, the T cell depleted immature hematopoietic cells comprise less than 1×10⁶ CD8⁺ TCRα/β⁻ cells per kilogram ideal body weight of the subject.

According to one embodiment, the immature hematopoietic cells are depleted of B cells.

According to an embodiment, the immature hematopoietic cells are depleted of B cells (CD19⁺ and/or CD20⁺ cells).

According to an embodiment, the immature hematopoietic cells comprise less than 1×10⁴-1×10⁶ CD19⁺ and/or CD20⁺ cells (e.g. 1×10⁵-50×10⁵ CD19⁺ and/or CD20⁺ cells) per kilogram ideal body weight of the subject.

According to an embodiment, the immature hematopoietic cells comprise less than about 50×10⁵ CD19⁺ and/or CD20⁺ cells, 40×10⁵ CD19⁺ and/or CD20⁺ cells, 30×10⁵ CD19⁺ and/or CD20⁺ cells, 20×10⁵ CD19⁺ and/or CD20⁺ cells, 10×10⁵ CD19⁺ and/or CD20⁺ cells, 9×10⁵ CD19⁺ and/or CD20⁺ cells, 8×10⁵ CD19⁺ and/or CD20⁺ cells, 7×10⁵ CD19⁺ and/or CD20⁺ CD19⁺ and/or CD20⁺ cells, 6×10⁵ CD19⁺ and/or CD20⁺ cells, 5×10⁵ CD19⁺ and/or CD20⁺ cells, 4×10⁵ CD19⁺ and/or CD20⁺ cells, 3×10⁵ CD19⁺ and/or CD20⁺ cells, 2×10⁵ CD19⁺ and/or CD20⁺ cells or 1×10⁵ CD19⁺ and/or CD20⁺ cells per kilogram ideal body weight of the subject.

According to a specific embodiment, the immature hematopoietic cells comprise less than 3×10⁵ CD19⁺ and/or CD20⁺ cells per kilogram ideal body weight of the subject.

Depletion of T cells, e.g. CD3⁺, CD2⁺, TCRα/β⁺, CD4⁺ and/or CD8⁺ cells, or B cells, e.g. CD19⁺ and/or CD20⁺ cells, may be carried out using any method known in the art, such as by eradication (e.g. killing) with specific antibodies or by affinity based purification e.g. such as by the use of magnetic cell separation techniques, FACS sorter and/or capture ELISA labeling.

Such methods are described herein and in THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 1992). For example, cells can be sorted by, for example, flow cytometry or FACS. Thus, fluorescence activated cell sorting (FACS) may be used and may have varying degrees of color channels, low angle and obtuse light scattering detecting channels, and impedance channels. Any ligand-dependent separation techniques known in the art may be used in conjunction with both positive and negative separation techniques that rely on the physical properties of the cells rather than antibody affinity, including but not limited to elutriation and density gradient centrifugation.

Other methods for cell sorting include, for example, panning and separation using affinity techniques, including those techniques using solid supports such as plates, beads and columns. Thus, biological samples may be separated by “panning” with an antibody attached to a solid matrix, e.g. to a plate.

Alternatively, cells may be sorted/separated by magnetic separation techniques, and some of these methods utilize magnetic beads. Different magnetic beads are available from a number of sources, including for example, Dynal (Norway), Advanced Magnetics (Cambridge, Mass., U.S.A.), Immuncon (Philadelphia, U.S.A.), Immunotec (Marseille, France), Invitrogen, Stem cell Technologies (U.S.A) and Cellpro (U.S.A). Alternatively, antibodies can be biotinylated or conjugated with digoxigenin and used in conjunction with avidin or anti-digoxigenin coated affinity columns.

According to one embodiment, cells may be processed on CliniMACS® column (available from Miltenyi Biotec).

According to an embodiment, different depletion/separation methods can be combined, for example, magnetic cell sorting can be combined with FACS, to increase the separation quality or to allow sorting by multiple parameters.

According to a specific embodiment, T cell depleted immature hematopoietic cells are obtained by a method comprising collecting mobilized PBMCs from a donor subject (e.g. the same donor subject from which non-mobilized PBMCs are collected for generation of non-GVHD inducing anti-third party cells, as discussed below).

According to one embodiment, mobilization is effected by G-CSF.

According to one embodiment, mobilization is effected by G-CSF and plerixafor.

According to a specific embodiment, the collection of mobilized PBMCs is obtained in a single collection.

According to a specific embodiment, the collection of the mobilized PBMCs is obtained in two, three, four, five or more daily collection, e.g. three daily collections (e.g. on consequent days or within a few days apart).

According to one embodiment, enrichment of CD34⁺ expressing cells is effected by incubating the PBMCs with a CD34 binding agent.

According to a specific embodiment, the CD34 binding agent is an antibody, e.g. a monoclonal antibody.

According to a specific embodiment, the CD34 monoclonal antibody is conjugated to magnetic particles, e.g. super-paramagnetic particles.

According to a specific embodiment, the CD34⁺ labeled cells are selected by magnetic separation techniques (as discussed in detail hereinabove).

According to a specific embodiment, the CD34 magnetically labeled cells (i.e. CD34⁺ expressing cells) are retained by the separation column (i.e. positive selection) and the CD34⁻ cells are removed. The CD34⁺ cells are then released from the column and collected.

According to one embodiment, depletion of T cells is effected by incubating the PBMCs with a CD3, CD2, TCRα/β, CD4 and/or CD8 binding agent.

According to one embodiment, depletion of B cells is effected by incubating the PBMCs with a CD19 and/or CD20 binding agent.

According to a specific embodiment, the CD3, CD2, TCRα/β, CD4, CD8, CD19 and/or CD20 binding agent is an antibody, e.g. monoclonal antibody.

According to a specific embodiment, the CD3, CD2, TCRα/β, CD4, CD8, CD19 and/or CD20 monoclonal antibody is conjugated to magnetic particles, e.g. super-paramagnetic particles.

According to one embodiment, the CD3, CD2, TCRα/β, CD4, CD8, CD19 and/or CD20 labeled cells are selected by magnetic separation techniques (as discussed in detail hereinabove).

According to a specific embodiment, the CD3, CD2, TCRα/β, CD4, CD8, CD19 and/or CD20 magnetically labeled cells (i.e. CD3, CD2, TCRα/β, CD4, CD8, CD19 and/or CD20 expressing cells) are retained by the separation column (i.e. negative selection) and the CD3−, CD2−, TCRα/β−, CD4−, CD8−, CD19− and/or CD20− cells are collected.

According to one embodiment, the T cell depleted immature hematopoietic cells and the PBMCs used for generation of the non-GVHD inducing anti-third party cells (i.e. for generation of veto cells as discussed below) are obtained from the same donor subject.

Depending on the application, the method may be affected using donor cells (e.g. T cell depleted immature hematopoietic cells, PBMCs used for generation of the non-GVHD inducing anti-third party cells, and/or non-hematopoietic cells, as discussed below) which are syngeneic or non-syngeneic with the recipient subject (e.g. allogeneic).

As used herein, the term “syngeneic” cells refer to cells which are essentially genetically identical with the subject or essentially all lymphocytes of the subject. Examples of syngeneic cells include cells derived from the subject (also referred to in the art as an “autologous”), from a clone of the subject, or from an identical twin of the subject.

As used herein, the term “non-syngeneic” cells refer to cells which are not essentially genetically identical with the subject or essentially all lymphocytes of the subject, such as allogeneic cells or xenogeneic cells.

As used herein, the term “allogeneic” refers to cells which are derived from a donor subject who is of the same species as the recipient subject, but which is substantially non-clonal with the recipient subject. Typically, outbred, non-zygotic twin mammals of the same species are allogeneic with each other. It will be appreciated that an allogeneic cell may be HLA identical, partially HLA identical or HLA non-identical (i.e. displaying one or more disparate HLA determinant) with respect to the recipient subject.

According to one embodiment, the donor is a human being.

As used herein, the term “xenogeneic” refers to a cell which substantially expresses antigens of a different species relative to the species of a substantial proportion of the lymphocytes of the subject. Typically, outbred mammals of different species are xenogeneic with each other.

The present invention envisages that xenogeneic cells are derived from a variety of species. Thus, according to one embodiment, the cells may be derived from any mammal. Suitable species origins for the cells comprise the major domesticated or livestock animals and primates. Such animals include, but are not limited to, porcines (e.g. pig), bovines (e.g., cow), equines (e.g., horse), ovines (e.g., goat, sheep), felines (e.g., Felis domestica), canines (e.g., Canis domestica), rodents (e.g., mouse, rat, rabbit, guinea pig, gerbil, hamster), and primates (e.g., chimpanzee, rhesus monkey, macaque monkey, marmoset). Cells of xenogeneic origin (e.g. porcine origin) are preferably obtained from a source which is known to be free of zoonoses, such as porcine endogenous retroviruses. Similarly, human-derived cells or tissues are preferably obtained from substantially pathogen-free sources.

Thus, the source of the cells will be determined with respect to the intended use thereof and is well within the capability of one skilled in the art, especially in light of the detailed disclosure provided herein.

The immature hematopoietic cells (e.g. T cell depleted immature hematopoietic cells) of some embodiments of the invention may be transplanted into a subject using any method known in the art for cell transplantation, such as but not limited to, cell infusion (e.g. I.V.) or via an intraperitoneal route, as discussed in detail herein below.

The immature hematopoietic cells of some embodiments of the invention may be administered in a single infusion (e.g. on day 0), or in 2, 3, 4 or more infusions (e.g. on consequent days or within days apart, e.g. on days −1 and 0; or on days 0 and 1). Accordingly, the immature hematopoietic cells of some embodiments of the invention may be obtained in 2, 3, 4 or more daily collection, e.g. two daily collections (e.g. on consequent days or within a few days apart).

According to one embodiment, the immature hematopoietic cells (e.g. T cell depleted) are collected at any time prior to the planned transplant date. Such cells can be stored for future use (e.g. cryopreserved).

Following transplantation of the immature hematopoietic cells into the subject according to the present teachings, it is advisable, according to standard medical practice, to monitor the growth functionality and immuno-compatibility of the cells according to any one of various standard art techniques. For example, the cell numbers of immature hematopoietic cells can be monitored in a subject by standard blood and bone marrow tests (e.g. by FACS analysis).

As illustrated in the Examples section which follows, anti-third party central memory T cells are endowed with specific veto activity and can be used as graft facilitating cells in situations in which non-syngeneic (e.g. allogeneic) transplantation of hematopoietic progenitor cells is warranted.

According to one embodiment, the non-GVHD inducing anti-third party cells of some embodiments of the present invention may be used as adjuvant therapy for transplantation of immature hematopoietic cells (as described hereinabove).

According to one embodiment, the non-GVHD inducing anti-third party cells may be used to avoid graft rejection, graft versus host disease and/or to induce donor specific tolerance (i.e. of the immature hematopoietic cells).

Thus, according to one embodiment, the subject is administered a therapeutically effective amount of an isolated population of non-GVHD inducing anti-third party cells comprising cells having a central memory T-lymphocyte (Tcm) phenotype, the cells being tolerance inducing cells and capable of homing to the lymph nodes following transplantation.

The term “isolated” refers to cells which have been isolated from their natural environment (e.g., the human body).

According to one embodiment, a population of cells refers to a heterogeneous cell mixture.

The term “non-graft versus host disease” or “non-GVHD” as used herein refers to having substantially reduced or no graft versus host (GVH) inducing reactivity. Thus, the cells of some embodiments of the present invention are generated as to not significantly cause graft versus host disease (GVHD) as evidenced by survival, weight and overall appearance of the transplanted subject 30-120 days following transplantation. Methods of evaluating a subject for reduced GVHD are well known to one of skill in the art.

According to one embodiment, the cells of some embodiments of the present invention have at least 10%, at least 20%, at least 30%, at least 40%, at least 50%, at least 55%, at least 60%, at least 65%, at least 70%, at least 75%, at least 80%, at least 85%, at least 90%, at least 95% or even 100% reduced reactivity against a host relative to cells not generated according to the present teachings.

The phrase “central memory T-lymphocyte (Tcm) phenotype” as used herein refers to a subset of T cytotoxic cells which home to the lymph nodes. Cells having the Tcm phenotype, in humans, typically comprise a CD3⁺/CD8⁺/CD62L⁺/CD45RO⁺/CD45RA⁻ signature. It will be appreciated that Tcm cells may express all of the signature markers on a single cell or may express only part of the signature markers on a single cell. Determination of a cell phenotype can be carried out using any method known to one of skill in the art, such as for example, by Fluorescence-activated cell sorting (FACS) or capture ELISA labeling.

According to one embodiment, at least 20%, at least 25%, at least 30%, at least 35%, at least 40%, at least 50%, at least 55%, at least 60%, at least 65%, at least 70%, at least 75%, at least 80%, at least 85%, at least 90%, at least 95%, at least 99%, or even 100% of the isolated population of non-GVHD inducing anti-third party cells have the Tcm cell signature.

According to a specific embodiment, about 10-20%, about 10-30%, about 10-40%, about 10-50%, about 20-30%, about 20-40%, about 30-50%, about 40-60%, about 50-70%, about 60-80%, about 70-90%, about 80-100%, or about 90-100% of the isolated population of non-GVHD inducing anti-third party cells have the Tcm cell signature.

According to a specific embodiment, cells having the Tcm phenotype comprise 10-30% of the isolated population of non-GVHD inducing cells.

According to a specific embodiment, cells having the Tcm phenotype comprise 10-50% of the isolated population of non-GVHD inducing cells.

According to a specific embodiment, cells having the Tcm phenotype comprise 20-40% of the isolated population of non-GVHD inducing cells.

According to a specific embodiment, cells having the Tcm phenotype comprise 30-50% of the isolated population of non-GVHD inducing cells.

According to a specific embodiment, cells having the Tcm phenotype comprise 50-70% of the isolated population of non-GVHD inducing cells.

According to a specific embodiment, cells having the Tcm phenotype comprise 20% of the isolated population of non-GVHD inducing cells.

According to a specific embodiment, cells having the Tcm phenotype comprise 30% of the isolated population of non-GVHD inducing cells.

According to a specific embodiment, cells having the Tcm phenotype comprise 40% of the isolated population of non-GVHD inducing cells.

According to a specific embodiment, cells having the Tcm phenotype comprise 50% of the isolated population of non-GVHD inducing cells.

According to a specific embodiment, cells having the Tcm phenotype comprise 60% of the isolated population of non-GVHD inducing cells.

According to a specific embodiment, cells having the Tcm phenotype comprise 70% of the isolated population of non-GVHD inducing cells.

The non-GVHD inducing cells comprising a Tcm phenotype of the invention are also referred to herein as “Tcm cells”.

As mentioned, Tcm cells typically home to the lymph nodes following transplantation. According to some embodiments, the isolated population of cells of some embodiments of the present invention may home to any of the lymph nodes following transplantation, as for example, the peripheral lymph nodes and mesenteric lymph nodes. The homing nature of these cells allows them to exert their veto effect in a rapid and efficient manner.

The non-GVHD inducing cells comprising a Tcm phenotype of some embodiments of the present invention are tolerance-inducing cells.

The phrase “tolerance inducing cells” as used herein refers to cells which provoke decreased responsiveness of the recipient's cells (e.g. recipient's T cells) when they come in contact with the recipient's cells as compared to the responsiveness of the recipient's cells in the absence of administered tolerance inducing cells. Tolerance inducing cells include veto cells (i.e. T cells which lead to apoptosis of host T cells upon contact with same) as was previously described in PCT Publication Nos. WO 2001/049243 and WO 2002/102971.

The term “veto activity” relates to immune cells (e.g. donor derived T cells) which lead to inactivation of anti-donor recipient T cells upon recognition and binding to the veto cells. According to one embodiment, the inactivation results in apoptosis of the anti-donor recipient T cells.

The non-GVHD inducing cells comprising a Tcm phenotype of the invention are also referred to herein as “veto cells”.

The phrase “anti-third party cells” as used herein refers to T lymphocytes which are directed (by T cell recognition) against a third party antigen or antigens.

As used herein the phrase “third party antigen or antigens” refers to a soluble or non-soluble (such as membrane associated) antigen or antigens which are not present in either the donor or recipient, as depicted in detail infra.

For example, an antigen or antigens can be whole cells (e.g. live or dead cells), cell fractions (e.g. lysed cells), cell antigens (e.g. cell surface antigens/proteins), a protein extract, a purified protein (e.g. ovalbumin) or a synthetic peptide.

According to one embodiment, the third party antigen or antigens is a non-self-antigen, i.e. an antigen or antigens which the immune system of the donor does not recognize as a self-antigen.

According to one embodiment, the antigen or antigens are non-mammalian, e.g. are non-human (e.g. proteins or peptides of a non-human animal or microbe, e.g., of a bird, reptile, viral, bacterial, fungal origin).

According to one embodiment, the antigen or antigens comprise viral antigens.

Exemplary viral antigens include, but are not limited to, an antigen of Epstein-Barr virus (EBV), Adenovirus (Adv), cytomegalovirus (CMV), cold viruses, flu viruses, hepatitis A, B, and C viruses, herpes simplex, HIV, influenza, Japanese encephalitis, measles, polio, rabies, respiratory syncytial, rubella, smallpox, varicella zoster, rotavirus, West Nile virus, Polyomavirus (e.g. BK Virus), zika virus, parvovirus (e.g. parvovirus B19), varicella-zoster virus (VZV), and Herpes simplex virus (HSV).

As further particular examples of viral antigens, BK Virus antigens include, but are not limited to, BKV LT; BKV (capsid VP1), BKV (capsid protein VP2), BKV (capsid protein VP2, isoporm VP3), BKV (small T antigen); Adenovirus antigens include, but are not limited to, Adv-penton or Adv-hexon; CMV antigens include, but are not limited to, envelope glycoprotein B, CMV IE-1 and CMV pp65, UL28, UL32,UL36, UL40, UL48,UL55,UL84, UL94, UL99 UL103, UL151, UL153, US 29, US 32; EBV antigens include, but are not limited to, EBV LMP2, EBV BZLF1, EBV EBNA1, EBV P18, and EBV P23; hepatitis antigens include, but are not limited to, the S, M, and L proteins of hepatitis B virus, the pre-S antigen of hepatitis B virus, HBCAG DELTA, HBV HBE, hepatitis C viral RNA, HCV NS3 and HCV NS4; herpes simplex viral antigens include, but are not limited to, immediate early proteins and glycoprotein D; HIV antigens include, but are not limited to, gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; influenza antigens include, but are not limited to, hemagglutinin and neuraminidase; Japanese encephalitis viral antigens include, but are not limited to, proteins E, M-E, M-E-NS1, NS1, NS1-NS2A and 80% E; measles antigens include, but are not limited to, the measles virus fusion protein; rabies antigens include, but are not limited to, rabies glycoprotein and rabies nucleoprotein; respiratory syncytial viral antigens include, but are not limited to, the RSV fusion protein and the M2 protein; rotaviral antigens include, but are not limited to, VP7sc; rubella antigens include, but are not limited to, proteins E1 and E2; and varicella zoster viral antigens include, but are not limited to, gpl and gpll.

According to one embodiment, the antigen or antigens comprise viral peptides.

According to one embodiment, the vial peptides comprise 1-25, 1-20, 1-15, 1-10, 1-9, 1-8, 1-7, 1-6, 1-5, 1-4, 1-3, 1-2, 2-20, 2-10, 2-8, 2-6, 2-4, 3-20, 3-10, 3-9, 3-7, 3-5, 3-4, 4-20, 4-10, 4-8 or 4-6 types of viral peptides.

According to a specific embodiment, the vial peptides comprise 4-10 types of viral peptides (e.g. in a single formulation or in several formulations).

According to a specific embodiment, the vial peptides comprise 4-8 types of viral peptides (e.g. in a single formulation or in several formulations).

According to a specific embodiment, the vial peptides comprise 4-6 types of viral peptides (e.g. in a single formulation or in several formulations).

According to one embodiment, the vial peptides comprise 1, 2, 3, 4, 5, 6, 7, 8, 9, 10, 11, 12, 13, 14, 15, 16, 17, 18, 19 or 20 types of viral peptides (e.g. in a single formulation or in several formulations).

According to a specific embodiment, the vial peptides comprise 4 types of viral peptides (e.g. in a single formulation or in several formulations).

According to a specific embodiment, the vial peptides comprise 5 types of viral peptides (e.g. in a single formulation or in several formulations).

According to a specific embodiment, the vial peptides comprise 6 types of viral peptides (e.g. in a single formulation or in several formulations).

According to a specific embodiment, the vial peptides comprise peptides from a single organism (i.e. from one virus type).

According to a specific embodiment, the vial peptides comprise peptides from two or more organism (i.e. a mixture from 2, 3, 4, 5 or more virus types).

According to one embodiment, the viral peptides comprise a BK virus peptide.

According to a specific embodiment, the viral peptides comprise Epstein-Barr virus (EBV) peptide, a cytomegalovirus (CMV) peptide, a BK Virus peptide and an Adenovirus (Adv) peptide.

According to a specific embodiment, the viral peptides comprise at least one of EBV-LMP2, EBV-BZLF1, EBV-EBNA1, EBV-BRAF1, EBV-BMLF1, EBV-GP340/350 EBNA2, EBV-EBNA3a, EBV-EBNA3b, EBV-EBNA3c, CMV-pp65, CMV-IE-1, Adv-penton, Adv-hexon, BKV LT, BKV (capsid VP1), BKV (capsid protein VP2), BKV (capsid protein VP2, isoporm VP3), and BKV (small T antigen).

According to a specific embodiment, the viral peptides comprise at least one of AdV5 Hexon, hCMV pp65, EBV select (discussed below) and BKV LT.

Dedicated software can be used to analyze antigen sequences to identify immunogenic short peptides, i.e., peptides presentable in context of major histocompatibility complex (MHC) class I or MHC class II.

According to a specific embodiment, the antigen or antigens comprise a mixture of pepmixes which are overlapping peptide libraries (e.g. 15mers overlapping by 11 amino acids) spanning the entire protein sequence of three viruses: CMV, EBV, and Adeno (such pepmixes can be commercially bought e.g. from JPT Technologies, Berlin, Germany).

According to a specific embodiment, the viral peptides comprise “EBV select” i.e. a commercial product from Miltenyi Biotec comprising 43 MHC class 1 and class 2 restricted peptides from 13 different proteins from EBV (e.g. MACS GMP PepTivator® EBV Select, e.g. catalog no. 170-076-143). Additionally or alternatively, the viral peptides comprise “collection EBV” i.e., a commercial product from JPT have comprising a pepmix which includes peptides from 14 different EBV antigens. Additionally or alternatively, the viral peptides comprise PepMix™ BKV (capsid protein VP1), PepMix™ BKV (capsid protein VP2), PepMix™ BKV (capsid protein VP2, isoform VP3), PepMix™ BKV (large T antigen), PepMix™ BKV (small T antigen), commercially available from JPT.

According to another specific embodiment, the antigen or antigens comprise a mixture of seven pepmixes spanning EBV-LMP2, EBV-BZLF1, EBV-EBNA1, CMV-pp65, CMV-IE-1, Adv-penton and Adv-hexon at a concentration of e.g. 100 ng/peptide or 700 ng/mixture of the seven peptides.

According to one embodiment, the antigen or antigens comprise antigen or antigens of an infectious organism (e.g., bacterial, fungal organism) which typically affects immune comprised subjects, such as transplantation patients.

According to one embodiment, the antigen is a bacterial antigen, such as but not limited to, an antigen of anthrax; gram-negative bacilli, chlamydia, diptheria, Haemophilus influenza, Helicobacter pylori, malaria, Mycobacterium tuberculosis, pertussis toxin, pneumococcus, rickettsiae, staphylococcus, streptococcus and tetanus.

As further particular examples of bacterial antigens, anthrax antigens include, but are not limited to, anthrax protective antigen; gram-negative bacilli antigens include, but are not limited to, lipopolysaccharides; Haemophilus influenza antigens include, but are not limited to, capsular polysaccharides; diptheria antigens include, but are not limited to, diptheria toxin; Mycobacterium tuberculosis antigens include, but are not limited to, mycolic acid, heat shock protein 65 (HSP65), the 30 kDa major secreted protein and antigen 85A; pertussis toxin antigens include, but are not limited to, hemagglutinin, pertactin, FIM2, FIM3 and adenylate cyclase; pneumococcal antigens include, but are not limited to, pneumolysin and pneumococcal capsular polysaccharides; rickettsiae antigens include, but are not limited to, rompA; streptococcal antigens include, but are not limited to, M proteins; and tetanus antigens include, but are not limited to, tetanus toxin.

According to one embodiment, the antigen is a superbug antigen (e.g. multi-drug resistant bacteria). Examples of superbugs include, but are not limited to, Enterococcus faecium, Clostridium difficile, Acinetobacter baumannii, Pseudomonas aeruginosa, and Enterobacteriaceae (including Escherichia coli, Klebsiella pneumoniae, Enterobacter spp.).

According to one embodiment, the antigen is a fungal antigen. Examples of fungi include, but are not limited to, candida, coccidiodes, cryptococcus, histoplasma, leishmania, plasmodium, protozoa, parasites, schistosomae, tinea, toxoplasma, and Trypanosoma cruzi.

As further particular examples of fungal antigens, coccidiodes antigens include, but are not limited to, spherule antigens; cryptococcal antigens include, but are not limited to, capsular polysaccharides; histoplasma antigens include, but are not limited to, heat shock protein 60 (HSP60); leishmania antigens include, but are not limited to, gp63 and lipophosphoglycan; Plasmodium falciparum antigens include, but are not limited to, merozoite surface antigens, sporozoite surface antigens, circumsporozoite antigens, gametocyte/gamete surface antigens, protozoal and other parasitic antigens including the blood-stage antigen pf 155/RESA; schistosomae antigens include, but are not limited to, glutathione-S-transferase and paramyosin; tinea fungal antigens include, but are not limited to, trichophytin; toxoplasma antigens include, but are not limited to, SAG-1 and p30; and Trypanosoma cruzi antigens include, but are not limited to, the 75-77 kDa antigen and the 56 kDa antigen.

According to one embodiment, the antigen or antigens comprise antigens associated with a malignant disease (e.g. tumor antigens).

According to one embodiment, the antigen is an antigen (or part thereof, e.g. antigen epitope) expressed by tumor cells. According to one embodiment, the antigen (or part thereof) is derived from a protein expressed in a hematopoietic tissue (e.g. hematopoietic malignancy such as leukemia antigen) or expressed in a solid tumor (e.g. melanoma, pancreatic cancer, liver cancer, gastrointestinal cancer, etc.).

Examples of tumor antigens include, but are not limited to, A33, BAGE, Bcl-2, B cell maturation antigen (BCMA), BCR-ABL, β-catenin, cancer testis antigens (CTA e.g. MAGE-1, MAGE-A2/A3 and NY-ESO-1), CA 125, CA 19-9, CA 50, CA 27.29 (BR 27.29), CA 15-3, CD5, CD19, CD20, CD21, CD22, CD33, CD37, CD45, CD123, CEA, c-Met, CS-1, cyclin B1, DAGE, EBNA, EGFR, ELA2, ephrinB2, estrogen receptor, FAP, ferritin, folate-binding protein, GAGE, G250/CA IX, GD-2, GM2, gp75, gp100 (Pmel 17), HA-1, HA-2, HER-2/neu, HM1.24, HPV E6, HPV E7, hTERT, Ki-67, LRP, mesothelin, mucin-like cancer-associated antigen (MCA), MUC1, p53, PR1, PRAME, PRTN3, RHAMM (CD168), WT-1. Further tumor antigens are provided in Molldrem J. Biology of Blood and Marrow Transplantation (2006) 12:13-18; Alatrash G. and Molldrem J., Expert Rev Hematol. (2011) 4(1): 37-50; Renkvist et al., Cancer Immunol Immunother (2001) 50:3-15; van der Bruggen P, Stroobant V, Vigneron N, Van den Eynde B. Peptide database: T cell-defined tumor antigens. Cancer Immun (2013), www(dot)cancerimmunity(dot)org/peptide/; Rittenhouse, Manderino, and Hass, Laboratory Medicine (1985) 16(9) 556-560; all of which are incorporated herein by reference.

According to one embodiment, the antigen or antigens comprise a mixture of antigens (e.g. a mixture of antigens of one group of antigens as discussed, e.g. viral antigens; or a mixture of antigens from different groups of antigens, e.g. viral and bacterial antigens, viral and tumor antigens).

According to one embodiment, the antigen or antigens comprise a mixture of viral peptides and tumor peptides (e.g. in a single formulation or in several formulations).

According to one embodiment, the antigen or antigens comprise a mixture of viral peptides and bacterial peptides (e.g. in a single formulation or in several formulations).

According to one embodiment, the antigen or antigens comprise a mixture of viral peptides and fungal peptides (e.g. in a single formulation or in several formulations).

According to one embodiment, third party antigens (e.g. protein extracts, purified proteins or synthetic peptides) can be presented by cells (e.g., cell line) infected therewith or otherwise made to express these peptides or proteins.

It will be appreciated that antigen presenting cells may express all of the antigens on a single cell or may express only part of the antigens on a single cell. Moreover, different antigen presenting cells (e.g. in the same preparation) may express different antigens. Accordingly, the antigen presenting cells (e.g. dendritic cells) comprise a heterogeneous cell mixture.

Third party antigens can be presented on the cellular, viral or bacterial surfaces or derived and/or purified therefrom. Additionally, a viral or bacterial antigen can be displayed on an infected cell and a cellular antigen can be displayed on an artificial vehicle (e.g. liposome), on an artificial antigen presenting cell (e.g. leukemic or fibroblast cell line transfected with the third party antigen or antigens), on autologous presenting cells, or on non-autologous presenting cells.

According to some embodiments of the invention, the antigen or antigens (e.g. viral peptides) can be presented by genetically modified antigen presenting cells or artificial antigen presenting cells exhibiting MHC antigens (also referred to as human leukocyte antigen (HLA)) recognizable by the T cells e.g. memory CD8 T cells (e.g. cell line transfected with the antigen or antigens).

Thus, antigen presenting cells, cell lines, artificial vehicles (such as a liposome) or artificial antigen presenting cells (e.g. leukemic or fibroblast cell line transfected with the antigen or antigens), can be used to present short synthetic peptides fused or loaded thereto or to present protein extracts or purified proteins. Such short peptides, protein extracts or purified proteins may be viral-, bacterial-, fungal-, or tumor-antigen derived peptides or peptides representing any other antigen.

According to one embodiment, the third party cells are stimulatory cells selected from the group consisting of cells purified from peripheral blood lymphocytes (PBL), spleen or lymph nodes, cytokine-mobilized PBLs, in vitro expanded antigen-presenting cells (APC), in vitro expanded dendritic cells (DC) and artificial antigen presenting cells.

According to one embodiment, the antigen or antigens is presented by antigen presenting cells (e.g. dendritic cells) of the same origin as the PBMCs used for generation of the non-GVHD inducing anti-third party cells (i.e. for generation of veto cells as discussed below).

According to one embodiment, the third party cells comprise dendritic cells.

According to one embodiment, the third party cells comprise mature dendritic cells.

Methods of generating third party dendritic cells, which may be used as stimulatory cells for production of non-GVHD inducing anti-third party cells, are well known in the art. Thus, as a non-limiting example, peripheral blood mononuclear cells (PBMCs) may be obtained a cell donor. CD14⁺ expressing cells are then selected and cultured (e.g. in cell culture plates) using DC cell medium (e.g. Cellgro DC medium) supplemented with supplemented with cytokines and growth factors. Determination of cytokines and growth factors to be used is within the skill of a person of skill in the art. For example, the cell culture medium is supplemented with IL-4 (e.g. 200-2000 IU/mL, e.g. 1000 IU/mL) and GM-CSF (e.g. 1000-4000 IU/mL, e.g. 2000 IU/mL). The cell suspension is then seeded (e.g. in cell culture plates e.g. Cell Factory plates) and incubated for 12-36 hours, e.g. for 16-24 hours, e.g. for 24 hours, in at 37° C., 5% CO₂.

In order to induce maturation of the CD14⁺ expressing cells into antigen presenting cells (e.g. dendritic cells), the CD14+ enriched cell preparation is cultured in the presence of maturation factors. Determination of maturation factors to be used is within the skill of a person of skill in the art. Thus, according to one embodiment, the seeded (e.g. in cell culture plates, e.g. Cell Factory plates) CD14+ enriched cells are cultured in the presence of IL-4 (e.g. 200-2000 IU/mL, e.g. 1000 IU/mL), GM-CSF (e.g. 1000-4000 IU/mL, e.g. 2000 IU/mL), LPS (e.g. 10-100 ng/mL, e.g. 40 ng/mL), and IFN-γ (e.g. 50-500 IU/mL, e.g. 200 IU/mL) for 10-24 hours, e.g. for 14-18 hours, e.g. for 16 hours, in at 37° C., 5% CO₂.

After the culturing period, the antigen presenting cells (e.g. mature dendritic cells i.e. mDCs) are obtained from the cell culture. According to one embodiment, non-adherent cells are removed and the antigen presenting cells (i.e. adherent cells) are detached from the culture plates and are loaded with an antigen or antigens.

As used herein the phrase “loading” refers to the attachment of an antigen or antigens (e.g. peptides or proteins, as discussed above) to MHC peptides (e.g. MHC class I or II) on the surface of the antigen-presenting cell (APC, e.g. dendritic cell).

According to a specific embodiment, the third party cells comprise irradiated dendritic cells.

Thus, according to one embodiment, the DCs are irradiated with about 5-10 Gy, about 10-20 Gy, about 20-30 Gy, about 20-40 Gy, about 20-50 Gy, about 10-50 Gy. According to a specific embodiment, the DCs are irradiated with about 10-50 Gy (e.g. 30 Gy).

Utilizing cells, virally infected cells, bacteria infected cells, viral peptides presenting cells or bacteria peptides presenting cells as third party antigens is particularly advantageous since such third party antigens include a diverse array of antigenic determinants and as such direct the formation of anti-third party cells of a diverse population, which may further serve in faster reconstitution of T-cells in cases where such reconstitution is required, e.g., following lethal or sublethal irradiation or chemotherapy procedure.

Accordingly, according to one embodiment, the non-GVHD inducing anti-third party cells may be referred to as anti-viral Tcm cells, anti-bacterial Tcm cells, anti-tumor Tcm cells, etc. (i.e. according to the antigen or antigens used to generate these cells).

According to some embodiments, the non-GVHD inducing cells of some embodiments of the present invention comprising a Tcm phenotype may be non-genetically modified cells or genetically modified cells (e.g. cells which have been genetically engineered to express or not express specific genes, markers or peptides or to secrete or not secrete specific cytokines) depending on the application needed (e.g. the type of T cell mediated autoimmune disease to be treated). Such determinations are well within the ability of one of ordinary skill in the art.

Any method of producing anti-third party Tcm cells can be used in accordance with the present invention as was previously described in PCT Publication Nos. WO 2010/049935, WO 2012/032526, WO 2013/035099 and WO 2018/002924, incorporated herein by reference.

Thus, for example, anti-third party cells having the Tcm phenotype may be generated by a method comprising: (a) contacting peripheral blood mononuclear cells (PBMCs) with a third party antigen or antigens in a culture deprived of cytokines so as to allow enrichment of antigen reactive cells; and (b) culturing the cells resulting from step (a) in the presence of cytokines so as to allow proliferation of cells comprising the central memory T-lymphocyte (Tcm) phenotype.

According to one embodiment, the PBMCs in step (a) are contacted with a third party antigen or antigens in the absence of IL-21.

According to one embodiment, the PBMCs in step (a) are contacted with a third party antigen or antigens in the presence of IL-21.

According to one embodiment, the PBMCs in step (a) are contacted with a third party antigen or antigens in a culture deprived of cytokines supplemented with only IL-21.

According to one embodiment, the cells resulting from step (a) are cultured in an antigen free environment (e.g. without the addition of an antigen to the cell culture) in the presence of IL-15.

According to one embodiment, the cells resulting from step (a) are cultured in an antigen free environment (e.g. without the addition of an antigen to the cell culture) in the presence of IL-15, IL-21 and/or IL-7.

The anti-third party Tcm cells of the present invention are typically generated by first contacting peripheral blood mononuclear cells (PBMCs, e.g. syngeneic or non-syngeneic, e.g. of the same cell donor as the immature hematopoietic cells) with a third party antigen or antigens (such as described above) in a cytokine free culture (i.e., without the addition of cytokines), or in a culture supplemented with only IL-21. Such a culture condition enables survival and enrichment of only those cells which undergo stimulation and activation by the third party antigen or antigens (i.e. of antigen reactive cells) as these cells secrete cytokines (e.g. IL-2) which enable their survival (all the rest of the cells die under these culture conditions). This step is typically carried out for about 12-24 hours, about 12-36 hours, about 12-72 hours, 24-48 hours, 24-36 hours, about 24-72 hours, about 48-72 hours, 1-2 days, 2-3 days, 1-3 days, 2-4 days, 1-5 days, 2-5 days, 2-6 days, 1-7 days, 5-7 days, 2-8 days, 8-10 days or 1-10 days and allows enrichment of antigen reactive cells.

According to a specific embodiment, contacting PBMCs with a third party antigen or antigens (such as described above) is effected for 1-5 days (e.g. 3 days).

According to one embodiment, culture with an antigen or antigens is effected in the presence of IL-21. This step is typically carried out in the presence of about 0.001-3000 IU/ml, 0.01-3000 IU/ml, 0.1-3000 IU/ml, 1-3000 IU/ml, 10-3000 IU/ml, 100-3000 IU/ml, 1000-3000 IU/ml, 0.001-1000 IU/ml, 0.01-1000 IU/ml, 0.1-1000 IU/ml, 1-1000 IU/ml, 10-1000 IU/ml, 100-1000 IU/ml, 250-1000 IU/ml, 500-1000 IU/ml, 750-1000 IU/ml, 10-500 IU/ml, 50-500 IU/ml, 100-500 IU/ml, 250-500 IU/ml, 100-250 IU/ml, 0.1-100 IU/ml, 1-100 IU/ml, 10-100 IU/ml, 30-100 IU/ml, 50-100 IU/ml, 1-50 IU/ml, 10-50 IU/ml, 20-50 IU/ml, 30-50 IU/ml, 1-30 IU/ml, 10-30 IU/ml, 20-30 IU/ml, 10-20 IU/ml, 0.1-10 IU/ml, or 1-10 IU/ml IL-21.

According to a specific embodiment, the concentration of IL-21 is 50-150 IU/ml (e.g. 100 IU/ml).

The ratio of third party antigen or antigens (e.g. dendritic cell) to PBMCs is typically about 1:1 to about 1:20, such as about 1:2 to about 1:10 such as about 1:4, about 1:6, about 1:8 or about 1:10. According to a specific embodiment, the ratio of third party antigen or antigens (e.g. dendritic cell) to PBMCs is about 1:2 to about 1:8 (e.g. 1:5).

Next, the anti-third party cells are cultured in the presence of IL-15 (e.g. in an antigen free environment), and optionally supplemented with IL-21 and/or IL-7, so as to allow proliferation of cells comprising the Tcm phenotype. This step is typically carried out for about 12-24 hours, about 12-36 hours, about 12-72 hours, 24-48 hours, 24-36 hours, about 24-72 hours, about 48-72 hours, 1-20 days, 1-15 days, 1-10 days, 1-5 days, 5-20 days, 5-15 days, 5-10 days, 1-2 days, 2-3 days, 1-3 days, 2-4 days, 2-5 days, 2-8 days, 2-10 days, 4-10 days, 4-8 days, 6-8 days, 8-10 days, 7-9 days, 7-11 days, 7-13 days, 7-15 days, 10-12 days, 10-14 days, 12-14 days, 14-16 days, 14-18 days, 16-18 days or 18-20 days.

According to a specific embodiment, the anti-third party cells are cultured in the presence of IL-15, and optionally supplemented with IL-21 and/or IL-7, in an antigen free environment (e.g. without the addition of an antigen) for about 7-11 days (e.g. 8 days).

According to one embodiment, culture with IL-15 is typically affected at a concentration of about 0.001-3000 IU/ml, 0.01-3000 IU/ml, 0.1-3000 IU/ml, 1-3000 IU/ml, 10-3000 IU/ml, 100-3000 IU/ml, 125-3000 IU/ml, 1000-3000 IU/ml, 0.001-1000 IU/ml, 0.01-1000 IU/ml, 0.1-1000 IU/ml, 1-1000 IU/ml, 10-1000 IU/ml, 100-1000 IU/ml, 125-1000 IU/ml, 250-1000 IU/ml, 500-1000 IU/ml, 750-1000 IU/ml, 10-500 IU/ml, 50-500 IU/ml, 100-500 IU/ml, 125-500 IU/ml, 250-500 IU/ml, 250-500 IU/ml, 125-250 IU/ml, 100-250 IU/ml, 0.1-100 IU/ml, 1-100 IU/ml, 10-100 IU/ml, 30-100 IU/ml, 50-100 IU/ml, 1-50 IU/ml, 10-50 IU/ml, 20-50 IU/ml, 30-50 IU/ml, 1-30 IU/ml, 10-30 IU/ml, 20-30 IU/ml, 10-20 IU/ml, 0.1-10 IU/ml, or 1-10 IU/ml IL-15. According to a specific embodiment the concentration of IL-15 is 100-150 IU/ml (e.g. 125 IU/ml).

According to one embodiment, supplementation of IL-15 with IL-21 is typically affected at a concentration of about 0.001-3000 IU/ml, 0.01-3000 IU/ml, 0.1-3000 IU/ml, 1-3000 IU/ml, 10-3000 IU/ml, 100-3000 IU/ml, 1000-3000 IU/ml, 0.001-1000 IU/ml, 0.01-1000 IU/ml, 0.1-1000 IU/ml, 1-1000 IU/ml, 10-1000 IU/ml, 100-1000 IU/ml, 250-1000 IU/ml, 500-1000 IU/ml, 750-1000 IU/ml, 10-500 IU/ml, 50-500 IU/ml, 100-500 IU/ml, 250-500 IU/ml, 100-250 IU/ml, 0.1-100 IU/ml, 1-100 IU/ml, 10-100 IU/ml, 30-100 IU/ml, 50-100 IU/ml, 1-50 IU/ml, 10-50 IU/ml, 20-50 IU/ml, 30-50 IU/ml, 1-30 IU/ml, 10-30 IU/ml, 20-30 IU/ml, 10-20 IU/ml, 0.1-10 IU/ml, or 1-10 IU/ml IL-21. According to a specific embodiment, the concentration of IL-21 is 50-150 IU/ml (e.g. 100 IU/ml).

According to one embodiment, supplementation of IL-15 with IL-7 is typically affected at a concentration of about 0.001-3000 IU/ml, 0.01-3000 IU/ml, 0.1-3000 IU/ml, 1-3000 IU/ml, 10-3000 IU/ml, 30-3000 IU/ml, 100-3000 IU/ml, 1000-3000 IU/ml, 0.001-1000 IU/ml, 0.01-1000 IU/ml, 0.1-1000 IU/ml, 1-1000 IU/ml, 10-1000 IU/ml, 30-1000 IU/ml, 100-1000 IU/ml, 250-1000 IU/ml, 500-1000 IU/ml, 750-1000 IU/ml, 10-500 IU/ml, 30-500 IU/ml, 50-500 IU/ml, 100-500 IU/ml, 250-500 IU/ml, 100-250 IU/ml, 0.1-100 IU/ml, 1-100 IU/ml, 10-100 IU/ml, 30-100 IU/ml, 50-100 IU/ml, 1-50 IU/ml, 10-50 IU/ml, 20-50 IU/ml, 30-50 IU/ml, 1-30 IU/ml, 10-30 IU/ml, 20-30 IU/ml, 10-20 IU/ml, 0.1-10 IU/ml, or 1-10 IU/ml IL-7. According to a specific embodiment the concentration of IL-7 is 10-50 IU/ml (e.g. 30 IU/ml).

The present inventors have collected through laborious experimentation and screening a number of criteria which may be harnessed towards to improving the proliferation of anti-third party cells comprising a central memory T-lymphocyte (Tcm) phenotype being devoid of graft versus host (GVH) reactive cells and/or being enhanced for anti-disease (e.g. GVL) reactive cells.

According to one embodiment, the PBMCs are depleted of CD4⁺ cells (e.g. T helper cells) and/or CD56⁺ cells (e.g. NK cells) prior to contacting with a third party antigen or antigens.

Depletion of CD4⁺ and/or CD56⁺ cells may be carried out using any method known in the art, such as by affinity based purification (e.g. such as by the use of MACS® beads, FACS sorter and/or capture ELISA labeling). Such a step may be beneficial in order to increase the purity of the CD8⁺ cells within the culture (i.e. eliminate other lymphocytes within the cell culture e.g. T CD4⁺ cells or NK cells) or in order to increase the number of CD8⁺ T cells.

According to one embodiment, the PBMCs comprise CD8⁺ T cells.

According to one embodiment, the PBMCs are selected for CD45RA⁺ and/or CD45RO⁻ cells (i.e. naïve T cells) prior to contacting with a third party antigen or antigens.

Selection of naïve CD8⁺ T cells may be effected by selection of cells expressing CD45RA⁺ and/or cells expressing CD45RO⁻ and may be carried out using any method known in the art, such as by affinity based purification (e.g. such as by the use of MACS® beads, FACS sorter and/or capture ELISA labeling).

According to one embodiment, the PBMCs comprise naïve CD8⁺ T cells.

According to one embodiment, the naïve T cells comprise a CD8⁺CD45RO⁻ phenotype.

According to one embodiment, the naïve T cells comprise a CD8⁺CD45RA⁺ phenotype.

According to another embodiment, the naïve T cells comprise a CD8⁺CD45RO⁻CD45RA⁺ phenotype.

According to a specific embodiment, when naïve T cells are used, the first step of culturing with an antigen or antigens is typically affected for 1-5 days (e.g. 3 days) and culturing in the presence of IL-15 (in an antigen free environment) is typically affected for 6-12 days (e.g. 8 days).

Alternatively, the PBMCs are selected for CD45RA⁻ cells and/or CD45RO⁺ (i.e. memory T cells) prior to contacting with a third party antigen or antigens.

The term “memory T cells” as used herein refers to a subset of T lymphocytes which have previously encountered and responded to an antigen, also referred to as antigen experienced T cells.

Selection of memory CD8⁺ T cells may be effected by selection of cells expressing CD45RA⁻ and/or cells expressing CD45RO⁺ and may be carried out using any method known in the art, such as by affinity based purification (e.g. such as by the use of MACS® beads, FACS sorter and/or capture ELISA labeling).

According to one embodiment, the PBMCs comprise memory CD8⁺ T cells.

According to one embodiment, the selection is carried out so as to obtain a cell fraction comprising CD8⁺ T cells of which at least about 10%, 20%, 30%, 40%, 50%, 60%, 70% or more are memory T cells.

According to one embodiment, the memory T cells comprise a CD8⁺CD45RO⁺ phenotype.

According to another embodiment, the memory T cells comprise a CD8⁺CD45RA⁻ phenotype.

According to another embodiment, the memory T cells comprise a CD8⁺CD45RO⁺CD45RA⁻ phenotype.

According to a specific embodiment, when memory T cells are used, the first step of culturing with an antigen or antigens is typically affected for 1-5 days (e.g. 3 days) and culturing in the presence of IL-15 (e.g. in an antigen free environment) is typically affected for 3-10 days (e.g. 6 days).

An additional step which may be carried out in accordance with the present teachings include culturing the PBMCs cells with a third party antigen or antigens in the presence of IL-15, and optionally supplemented with IL-21 and/or IL-7 prior to removing the third party antigen or antigens from the cell culture (i.e. prior to generating an antigen free environment). This step is typically carried out for about 12-24 hours, about 12-36 hours, about 12-72 hours, 24-48 hours, 24-36 hours, about 24-72 hours, about 48-72 hours, 1-2 days, 2-3 days, 1-3 days, 2-4 days, 1-5 days or 2-5 days, and is effected at the same doses of IL-21, IL-15 and IL-7 indicated above. According to a specific embodiment, culturing the PBMCs cells with a third party antigen or antigens in the presence of IL-21, IL-15 and IL-7 is carried out for 12 hours to 4 days (e.g. 1-2 days).

Additionally or alternatively, an additional two step process which allows selection and isolation of activated cells may be carried out. Such a selection step aids in removal of potential host reactive T cells (e.g. alloreactive cells) in situations where the PBMCs are non-syngeneic with respect to the subject.

Thus, isolating activated cells may be carried out in a two stage approach. In the first stage activated cells are selected before culturing the cells in the presence of IL-15. This first stage is typically carried out after the initial contacting of the PBMCs with a third party antigen or antigens. This selection process picks only those cells which were activated by the third party antigen (e.g. express activation markers as described below) and is typically affected about 12-24 hours, about 24-36 hours, about 12-36 hours, about 36-48 hours, about 12-48 hours, about 48-60 hours, about 12-60 hours, about 60-72 hours, about 12-72 hours, about 72-84 hours, about 12-84 hours, about 84-96 hours, about 12-96 hours, after the initial contacting of the PBMCs with a third party antigen or antigens. According to a specific embodiment, the selection process is effected about 12-24 hours (e.g. 14 hours) after the initial contacting of the PBMCs with a third party antigen or antigens.

Isolating activated cells may be effected by affinity based purification (e.g. such as by the use of MACS® beads, FACS sorter and/or capture ELISA labeling) and may be effected towards any activation markers including cell surface markers such as, but not limited to, CD69, CD44, CD25, CFSE, CD137 or non-cell surface markers such as, but not limited to, IFN-γ and IL-2. Isolating activated cells may also be effected by morphology based purification (e.g. isolating large cells) using any method known in the art (e.g. by FACS). Typically, the activated cells are also selected for expression of CD8⁺ cells. Furthermore, any combination of the above methods may be utilized to efficiently isolate activated cells.

According to an embodiment of the present invention, selecting for activated cells is effected by selection of CD137⁺ and/or CD25⁺ cells.

The second stage of isolation of activated cells is typically carried out at the end of culturing (i.e. after culturing with IL-15 without the addition of an antigen). This stage depletes alloreactive cells by depletion of those cells which were activated following contacting of the central memory T-lymphocyte (Tcm) with irradiated host antigen presenting cells (APCs e.g. dendritic cells). As mentioned above, isolating activated cells may be effected by affinity based purification (e.g. such as by the use of MACS® beads, FACS sorter and/or capture ELISA labeling) and may be effected towards any activation markers including cell surface markers such as, but not limited to, CD69, CD44, CD25, CFSE, CD137 or non-cell surface markers such as, but not limited to, IFN-γ and IL-2.

According to an embodiment of the present invention, depleting the alloreactive cells is effected by depletion of CD137⁺ and/or CD25⁺ cells and/or IFNγ-capture.

According to one embodiment, the isolated population of non-GVHD inducing anti-third party cells generated by the present methods typically comprises 20-100% Tcm cells.

According to one embodiment, the isolated population of non-GVHD inducing anti-third party cells generated by the present methods comprise at least about 20%, at least about 30%, at least about 40%, at least about 50%, at least about 60%, at least about 70%, at least about 80%, at least about 90%, or more, Tcm cells.

According to a specific embodiment, the isolated population of non-GVHD inducing anti-third party cells generated by the present methods comprise at least about 30% Tcm cells.

According to a specific embodiment, the isolated population of non-GVHD inducing anti-third party cells generated by the present methods comprise at least about 40% Tcm cells.

According to a specific embodiment, the isolated population of non-GVHD inducing anti-third party cells generated by the present methods comprise at least about 50% Tcm cells.

According to a specific embodiment, the isolated population of non-GVHD inducing anti-third party cells generated by the present methods comprise at least about 60% Tcm cells.

According to a specific embodiment, the isolated population of non-GVHD inducing anti-third party cells generated by the present methods comprise at least about 70% Tcm cells.

Thus, the anti-third party cells having a central memory T-lymphocyte (Tcm) phenotype of the invention are not naturally occurring and are not a product of nature. These cells are typically produced by ex-vivo manipulation (i.e. exposure to a third party antigen or antigens in the absence or presence of specific cytokines).

According to one embodiment, the immature hematopoietic cells and the non-GVHD inducing anti-third party cells having the Tcm phenotype are obtained from the same donor (e.g. human being).

According to one embodiment, the immature hematopoietic cells and the non-GVHD inducing anti-third party cells having the Tcm phenotype are obtained from different donors (e.g. human beings).

According to one embodiment, the immature hematopoietic cells and the non-GVHD inducing anti-third party cells having the Tcm phenotype (i.e. veto cells) are administered concomitantly, i.e. co-administrated (e.g. at the same time or on the same day, e.g. within 12-24 hours).

Alternatively, the non-GVHD inducing anti-third party cells having the Tcm phenotype (i.e. veto cells) may be administered following transplantation of immature hematopoietic cells.

According to a specific embodiment, the anti-third party cells having the Tcm phenotype may be administered 1-30 days (e.g. 1-25 days, e.g. 1-20 days, e.g. 1-10, e.g. 4-10 days, e.g. 1-5 days) following transplantation of immature hematopoietic cells.

According to a specific embodiment, the anti-third party cells having the Tcm phenotype may be administered 1, 2, 3, 4, 5, 6, 7, 8, 9, 10, 11, 12, 13, 14, 15, 16, 17, 18, 19, 20, 21, 25 or 30 days (e.g. 3 days, 5 days, 7 days, 10 days) following transplantation of immature hematopoietic cells.

According to a specific embodiment, the anti-third party cells having the Tcm phenotype may be administered 8 days following transplantation of immature hematopoietic cells.

According to a specific embodiment, the anti-third party cells having the Tcm phenotype may be administered 7 days following transplantation of immature hematopoietic cells.

According to a specific embodiment, the anti-third party cells having the Tcm phenotype may be administered 6 days following transplantation of immature hematopoietic cells.

According to a specific embodiment, the anti-third party cells having the Tcm phenotype may be administered 5 days following transplantation of immature hematopoietic cells.

According to one embodiment, the non-GVHD inducing anti-third party Tcm cells may be administered to the subject in a single dose. Alternatively, the non-GVHD inducing anti-third party Tcm cells may be administered to the subject in two or more doses (e.g. three, four, five times or more).

Such a determination is well within the capability of one of skill in the art.

As T cell mediated autoimmune diseases typically cause tissue or organ damage, embodiments of the invention contemplate transplanting to a subject non-hematopoietic cells capable of regenerating the damaged tissue or organ.

As used herein, the term “non-hematopoietic cells” refers to bodily cells which are not of the hematopoietic lineage. Such cells include differentiated cells as well as progenitor cells and stem cells.

As used herein, the phrase “differentiated cells” refers to terminally differentiated cells. Exemplary cells which may be transplanted according to the present teachings include, but are not limited to, liver, pancreas, spleen, kidney, heart, lung, skin, intestine, fallopian tubes, ovarian, nerve or brain cells.

According to one embodiment, the differentiated cells are obtained from an adult tissue (i.e. a tissue of an organism at any time after birth).

According to one embodiment, the differentiated cells are obtained from a fetal tissue (as discussed below).

According to one embodiment of the invention, the non-hematopoietic cells comprise progenitor or stem cells.

As used herein, the phrase “stem cells” refers to cells which can differentiate into other cell types having a particular, specialized function (e.g., fully differentiated cells). Examples include but are not limited to totipotent, pluripotent, or multipotent cells.

The totipotent stem cells give rise to “progenitor cells” more differentiated than the totipotent cells. These cells are capable of differentiating into specific cell lineages, e.g. endothelial lineage, epithelial lineage or mesenchymal lineage.

According to one embodiment, the progenitor cells comprise hepatic progenitor cells, pancreatic progenitor cells, cardiac progenitor cells, brain progenitor cells, nerve progenitor cells, nephric progenitor cells, ovarian progenitor cells, spleen progenitor cells or pulmonary progenitor cells.

Depending on the application and available sources, the non-hematopoietic cells of the present invention may be obtained from a prenatal organism, postnatal organism, an adult or a cadaver donor. Such determinations are well within the ability of one of ordinary skill in the art.

According to one embodiment, cells may be obtained from an organ or tissue.

According to one embodiment, the organ or tissue is from a fetal organism. The fetal organism may be of any of a human or xenogeneic origin (e.g. porcine) and at any stage of gestation. Such a determination is in the capacity of one of ordinary skill in the art.

According to one embodiment, the fetal organ or tissue comprises a fetal pulmonary tissue, a fetal pancreatic tissue, a fetal nephric tissue, a fetal hepatic tissue, a fetal cardiac tissue, a fetal nerve tissue, a fetal brain tissue, a fetal spleen tissue, a fetal intestinal tissue, a fetal skin tissue, a fetal fallopian tube tissue, and a fetal ovarian tissue.

Various methods may be employed to obtain an organ or tissue from a fetal organism. Thus, for example, obtaining a tissue (e.g. liver or pancreas tissue) may be effected by harvesting the tissue from a developing fetus, e.g. by a surgical procedure, at a stage of gestation corresponding to human 14-24 weeks of gestation. It will be understood by those of skill in the art that the gestational stage of an organism is the time period elapsed following fertilization of the oocyte generating the organism.

Likewise, various methods may be employed to obtain an organ or tissue from an adult organism (e.g. live or cadaver). Thus, for example, obtaining a tissue (e.g. liver or pancreas tissue) may be effected by harvesting the tissue from an organ donor by a surgical procedure e.g. laparotomy or laparoscopy.

Alternatively, a tissue may be obtained by in-vitro or ex-vivo culture of cells, organs or tissues. Such controlled in-vitro differentiation of cells, tissues or organs is routinely performed, for example, using culturing of embryonic stem cell lines to generate cultures containing cells/tissues/organs of desired lineages. It will be appreciated that the non-hematopoietic cells of the invention may be of fresh or frozen (e.g., cryo-preserved) preparations.

According to one embodiment, the non-hematopoietic cells are in suspension. Accordingly, the non-hematopoietic cells are isolated from their natural environment (e.g., the human body) and are extracted from tissue/organ while maintaining viability but do not maintain a tissue structure (i.e., no vascularized tissue structure) such that they may be injectable (such as by intravenous administration). According to a specific embodiment the cells in suspension are not attached to a solid support.

According to one embodiment, the immature hematopoietic cells and/or the non-GVHD inducing anti-third party cells having the Tcm phenotype of the invention can be used to support transplantation of whole or partial organs or tissues. Exemplary organs or tissues which may be transplanted according to the present teachings include, but are not limited to, pulmonary organ or tissue, pancreatic organ or tissue, nephric organ or tissue, hepatic organ or tissue, cardiac organ or tissue, brain organ or tissue, spleen organ or tissue, intestinal organ or tissue, skin tissue, fallopian tube organ or tissue, ovarian organ or tissue. It will be appreciated that the organ or tissue may be obtained from a fetal or adult organism (as discussed above).

According to a specific embodiment, the organ or tissue is syngeneic or non-syngeneic with the recipient subject (e.g. allogeneic).

According to a specific embodiment, the organ or tissue is syngeneic with respect to the immature hematopoietic cells and/or the non-GVHD inducing anti-third party cells having the Tcm phenotype (i.e. veto cells).

According to a specific embodiment, the organ or tissue, the immature hematopoietic cells and the non-GVHD inducing anti-third party cells having the Tcm phenotype (i.e. veto cells) are from the same donor (e.g. human being).According to a specific embodiment, when the T cell mediated autoimmune disease involves the pancreas (e.g. autoimmune type 1 diabetes mellitus (T1DM)), the transplant further comprises a pancreatic cell, tissue or organ graft.

According to a specific embodiment, when the T cell mediated autoimmune disease involves the liver (e.g. autoimmune hepatitis), the transplant further comprises a liver cell, tissue or organ graft.

According to a specific embodiment, when the T cell mediated autoimmune disease involves the kidney (e.g. Glomerulonephritis), the transplant further comprises a kidney cell, tissue or organ graft.

According to a specific embodiment, when the T cell mediated autoimmune disease involves the intestines (e.g. Crohn's disease, ulcerative colitis (UC)), the transplant further comprises an intestine cell, tissue or organ graft.

According to a specific embodiment, when the T cell mediated autoimmune disease involves the central nervous system (e.g. multiple sclerosis), the transplant further comprises a nerve cell or tissue graft.

The immature hematopoietic cells and/or the non-GVHD inducing anti-third party cells having the Tcm phenotype and/or the non-hematopoietic cells, tissues or organs of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the immature hematopoietic cells and/or the non-GVHD inducing anti-third party cells having the Tcm phenotype and/or the non-hematopoietic cells accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Administering the immature hematopoietic cells and/or the non-GVHD inducing anti-third party cells having the Tcm phenotype and/or the non-hematopoietic cells into the subject may be effected in numerous ways, depending on various parameters, such as, for example, the cell type; the type, stage or severity of the recipient's disease (e.g. T cell mediated autoimmune disease); the physical or physiological parameters specific to the subject; and/or the desired therapeutic outcome.

For example, depending on the application and purpose, administration of the immature hematopoietic cells and/or the non-GVHD inducing anti-third party cells having the Tcm phenotype and/or the non-hematopoietic cells may be effected by a route selected from the group consisting of intratracheal, intrabronchial, intraalveolar, intravenous, intraperitoneal, intranasal, subcutaneous, intramedullary, intrathecal, intraventricular, intracardiac, intramuscular, intraserosal, intramucosal, transmucosal, transnasal, rectal and intestinal.

According to one embodiment, administering is effected by an intravenous route.

Alternatively, administration to the subject of the immature hematopoietic cells and/or the non-GVHD inducing anti-third party cells having the Tcm phenotype and/or the non-hematopoietic cells, tissues or organs may be effected by administration thereof into various suitable anatomical locations so as to be of therapeutic effect. Thus, depending on the application and purpose, the cells may be administered into a homotopic anatomical location (a normal anatomical location for the organ or tissue type of the cells), or into an ectopic anatomical location (an abnormal anatomical location for the organ or tissue type of the cells).

Accordingly, depending on the application and purpose, the cells, tissues or organs implanted (e.g. transplanted) under the renal capsule, or into the kidney, the testicular fat, the sub cutis, the omentum, the portal vein, the liver, the spleen, the heart cavity, the heart, the chest cavity, the lung, the pancreas, the skin and/or the intra-abdominal space.

For example, for the treatment of a T cell mediated autoimmune disease involving the liver (e.g. autoimmune hepatitis), non-hematopoietic cells can be transplanted into the liver, the portal vein, the renal capsule, the sub-cutis, the omentum, the spleen, and the intra-abdominal space. Similarly, for the treatment of a T cell mediated autoimmune disease involving the pancreas (e.g. autoimmune type 1 diabetes mellitus (T1DM)) transplanting non-hematopoietic cells according to the present invention can be effected by transplanting the cells into the portal vein, the liver, the pancreas, the testicular fat, the sub-cutis, the omentum, an intestinal loop (the subserosa of a U loop of the small intestine) and/or the intra-abdominal space. Likewise, transplantation of non-hematopoietic cells can be carried out for the purpose of treating recipients suffering from, for example, T cell mediated autoimmune disease involving the intestine, colon, stomach, brain and spinal cord (central nervous system).

Optionally, when transplanting the non-hematopoietic cells, tissues or organs of the present invention into a subject having a defective or damaged organ, the failed organ according to an embodiment is first at least partially removed from the subject so as to enable optimal development of the transplant, and structural/functional integration thereof with the anatomy/physiology of the subject.

Transplantation of non-hematopoietic cells, tissues or organs may be effected concomitantly with or following treatment with the hematopoietic cells and/or the non-GVHD inducing anti-third party cells having the Tcm phenotype. For example, non-hematopoietic cells, tissues or organs may be administered e.g. 1-30 days, 30-60 days, 60-90 days, 90-120 days, 120-180 days, e.g. 6-12 months, e.g. 12-24 months or more following treatment with the hematopoietic cells and/or the non-GVHD inducing anti-third party cells having the Tcm phenotype. Such determinations are well within the ability of one of ordinary skill in the art and depend on the disease to be treated, disease severity, the age of the subject, etc.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (the immature hematopoietic cells and/or the non-GVHD inducing anti-third party cells having the Tcm phenotype and/or the non-hematopoietic cells) effective to prevent, alleviate or ameliorate symptoms of a disorder (e.g., T cell mediated autoimmune disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

For example, the number of non-GVHD inducing anti-third party Tcm cells (i.e. veto cells) infused to a recipient should be more than 1×10⁴/Kg ideal body weight. The number of non-GVHD inducing anti-third party Tcm cells infused to a recipient should typically be in the range of 1×10³/Kg ideal body weight to 1×10⁴/Kg ideal body weight, range of 1×10⁴/Kg ideal body weight to 1×10⁵/Kg ideal body weight, range of 1×10⁴/Kg ideal body weight to 1×10⁶/Kg ideal body weight, range of 1×10⁴/Kg ideal body weight to 1×10⁷/Kg ideal body weight, range of 1×10⁴/Kg ideal body weight to 1×10⁸/Kg ideal body weight, range of 1×10³/Kg ideal body weight to 1×10⁵/Kg ideal body weight, range of 1×10⁴/Kg ideal body weight to 1×10⁶/Kg ideal body weight, range of 1×10⁶/Kg ideal body weight to 1×10⁷/Kg ideal body weight, range of 1×10⁵/Kg ideal body weight to 1×10⁷/Kg ideal body weight, range of 1×10⁶/Kg ideal body weight to 1×10⁸/Kg ideal body weight, or range of 1×10⁶/Kg ideal body weight to 1×10⁹/Kg ideal body weight.

According to a specific embodiment, the number of non-GVHD inducing anti-third party

Tcm cells infused to a recipient should be in the range of 0.5×10⁶/Kg ideal body weight to 1×10⁸/Kg ideal body weight.

According to a specific embodiment, the number of non-GVHD inducing anti-third party Tcm cells infused to a recipient should be in the range of 1×10⁵ CD8⁺ cells/Kg ideal body weight to 1×10⁸ CD8⁺ cells/Kg ideal body weight.

According to a specific embodiment, the number of non-GVHD inducing anti-third party Tcm cells infused to a recipient should comprise at least 0.5×10⁶ CD8⁺ cells/Kg ideal body weight.

According to a specific embodiment, the number of non-GVHD inducing anti-third party Tcm cells infused to a recipient should comprise at least 1×10⁶ CD8⁺ cells/Kg ideal body weight.

According to a specific embodiment, the number of non-GVHD inducing anti-third party Tcm cells infused to a recipient should comprise at least 2.5×10⁶ CD8⁺ cells/Kg ideal body weight.

According to a specific embodiment, the number of non-GVHD inducing anti-third party Tcm cells infused to a recipient should comprise at least 5×10⁶ CD8⁺ cells/Kg ideal body weight.

According to a specific embodiment, the number of non-GVHD inducing anti-third party Tcm cells infused to a recipient should comprise at least 7.5×10⁶ CD8⁺ cells/Kg ideal body weight.

According to a specific embodiment, the number of non-GVHD inducing anti-third party Tcm cells infused to a recipient should comprise at least 10×10⁶ CD8⁺ cells/Kg ideal body weight.

Therapeutically effective amounts of immature hematopoietic cells, e.g. T cell depleted immature hematopoietic cells, are discussed in detail hereinabove.

The term “ideal body weight” as used herein, refers to the measurement used clinically to adjust drug dosing, help estimate renal function and the pharmacokinetics (such as in obese patients).

The formula for estimating ideal body weight in (kg) is as follows:

Males: IBW=50 kg+2.3 kg for each inch over 5 feet.

Females: IBW=45.5 kg+2.3 kg for each inch over 5 feet.

Ideal body weight is discussed in detail in Peterson et al. [Am J Clin Nutr 2016; 103:1197-203], incorporated herein by reference.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

The amount of a composition to be administered will, of course, be dependent on the subject being treated, the severity of the affliction, the manner of administration, the judgment of the prescribing physician, etc.

Since non-syngeneic (e.g. allogeneic) cells are likely to induce an immune reaction when administered to the subject several approaches have been developed to reduce the likelihood of rejection of non-syngeneic cells. These include either suppressing the recipient immune system or encapsulating the non-autologous cells in immunoisolating, semipermeable membranes before transplantation. Alternatively, cells may be uses which do not express xenogenic surface antigens, such as those developed in transgenic animals (e.g. pigs).

Following transplantation of the immature hematopoietic cells and/or non-GVHD inducing anti-third party cells and/or non-hematopoietic cells, tissues or organs into the subject according to the present teachings, it is advisable, according to standard medical practice, to monitor the survival and functionality of the cells as well as development of graft rejection and/or graft versus host disease. Such methods are well known to a person of skill in the art. For example, the cell numbers of immature hematopoietic cells can be monitored in a subject by standard blood and bone marrow tests (e.g. by FACS analysis). Graft rejection or GVHD may be monitored by blood tests, physical examination (e.g. monitoring for skin rash, yellow discoloration, abdominal swelling, vomiting, diarrhea, abdominal cramps, nausea, increased dryness/irritation of the eyes) and biopsy (e.g. of the liver, lung). For example, the functionality of pancreatic cells may be monitored following transplantation by standard pancreas function tests (e.g. analysis of serum levels of insulin). Likewise, a liver cell transplant may be monitored following transplantation by standard liver function tests (e.g. analysis of serum levels of albumin, total protein, ALT, AST, and bilirubin, and analysis of blood-clotting time). Structural development of the non-hematopoietic cells may be monitored via computerized tomography, or ultrasound imaging.

Furthermore, following transplantation of the immature hematopoietic cells and/or non-GVHD inducing anti-third party cells into the subject according to the present teachings, it is advisable to monitor the reversal of T cell mediated autoimmune disease symptoms. For example, symptoms of Type 1 diabetes include, for example, being very thirsty, feeling hungry, fatigue, blurry eyesight, feeling numbness or feeling tingling in the feet, losing weight without trying, etc. Symptoms of Hashimoto's thyroiditis include, for example, enlarged neck or presence of goiter, fatigue, hair loss, intolerance to cold, small or shrunken thyroid gland, joint stiffness, unintentional weight gain, and swelling of the face. Symptoms of MS include, for example, fatigue, loss of balance, muscle spasms, numbness or abnormal sensation in any area, problems moving arms or legs, problems walking, problems with coordination and making small movements, tremor in one or more arms or legs, weakness in one or more arms or legs, vision loss, facial pain, hearing loss, slurred or difficult-to-understand speech, and trouble chewing and swallowing. These symptoms can be monitored by physical examination, ultrasound, MRI, blood tests, etc. Such determinations are well within the ability of one of ordinary skill in the art.

In order to treat T cell mediated autoimmune disease and to facilitate engraftment of the immature hematopoietic cells, the method may further comprise conditioning the subject under sublethal, lethal or supralethal conditions.

As used herein, the terms “sublethal”, “lethal”, and “supralethal”, when relating to conditioning of subjects of the present invention, refer to myelotoxic and/or lymphocytotoxic treatments which, when applied to a representative population of the subjects, respectively, are typically: non-lethal to essentially all members of the population; lethal to some but not all members of the population; or lethal to essentially all members of the population under normal conditions of sterility.

According to some embodiments of the invention, the sublethal, lethal or supralethal conditioning comprises a total body irradiation (TBI), total lymphoid irradiation (TLI, i.e. exposure of all lymph nodes, the thymus, and spleen), partial body irradiation (e.g. specific exposure of the lungs, kidney, brain etc.), myeloablative conditioning and/or non-myeloablative conditioning, e.g. with different combinations including, but not limited to, co-stimulatory blockade, chemotherapeutic agent and/or antibody immunotherapy. According to some embodiments of the invention, the conditioning comprises a combination of any of the above described conditioning protocols (e.g. chemotherapeutic agent and TBI, co-stimulatory blockade and chemotherapeutic agent, antibody immunotherapy and chemotherapeutic agent, etc).

According to one embodiment, the conditioning is effected by conditioning the subject under supralethal conditions, such as under myeloablative conditions (i.e. intensive conditioning regimen in which the bone marrow cells are destroyed).

Alternatively, the conditioning may be effected by conditioning the subject under lethal or sublethal conditions, such as by conditioning the subject under myeloreductive conditions or non-myeloablative conditions, respectively (i.e. reduced intensity conditioning which is a less aggressive conditioning regimen).

According to a specific embodiment, the conditioning comprises non-myeloablative conditioning (e.g. a reduced intensity conditioning regimen).

According to an embodiment, the reduced intensity conditioning is effected for up to 2 weeks (e.g. 1-10 or 1-7 days).

According to a specific embodiment, the non-myeloablative conditioning comprises a chemotherapeutic agent and TBI/TLI.

According to one embodiment, the TBI comprises an irradiation dose (e.g. single or fractionated irradiation dose) within the range of 0.5-1 Gy, 0.5-1.5 Gy, 0.5-2 Gy, 0.5-2.5 Gy, 0.5-5 Gy, 0.5-7.5 Gy, 0.5-10 Gy, 0.5-15 Gy, 1-1.5 Gy, 1-2 Gy, 1-2.5 Gy, 1-3 Gy, 1-3.5 Gy, 1-4 Gy, 1-4.5 Gy, 1-5 Gy, 1-5.5 Gy, 1-6 Gy, 1-7 Gy, 1-7.5 Gy, 1-10 Gy, 2-3 Gy, 2-4 Gy, 2-5 Gy, 2-6 Gy, 2-7 Gy, 2-8 Gy, 2-9 Gy, 2-10 Gy, 3-4 Gy, 3-5 Gy, 3-6 Gy, 3-7 Gy, 3-8 Gy, 3-9 Gy, 3-10 Gy, 4-5 Gy, 4-6 Gy, 4-7 Gy, 4-8 Gy, 4-9 Gy, 4-10 Gy, 5-6 Gy, 5-7 Gy, 5-8 Gy, 5-9 Gy, 5-10 Gy, 6-7 Gy, 6-8 Gy, 6-9 Gy, 6-10 Gy, 7-8 Gy, 7-9 Gy, 7-10 Gy, 8-9 Gy, 8-10 Gy, 10-12 Gy or 10-15 Gy.

According to a specific embodiment, the TBI comprises a single or fractionated irradiation dose within the range of 1-7.5 Gy.

According to a specific embodiment, the TBI comprises a single or fractionated irradiation dose within the range of 1-6 Gy.

According to a specific embodiment, the TBI comprises a single or fractionated irradiation dose within the range of 1-5 Gy.

According to a specific embodiment, the TBI comprises a single or fractionated irradiation dose of 6 Gy.

According to a specific embodiment, the TBI comprises a single or fractionated irradiation dose of 5 Gy.

According to a specific embodiment, the TBI comprises a single or fractionated irradiation dose of 4 Gy.

According to a specific embodiment, the TBI comprises a single or fractionated irradiation dose of 3 Gy.

According to an embodiment, TBI treatment is administered to the subject 1-10 days (e.g. 1-3 days, e.g. 1 day) prior to transplantation. According to one embodiment, the subject is conditioned once with TBI 1, 2, 3 or 4 days (e.g. 1 day) prior to transplantation. According to one embodiment, TBI is administered on the day of transplantation (i.e. day 0).

According to a specific embodiment, the TBI comprises a single or fractionated irradiation dose on days −3 to −1 (i.e. prior to transplantation).

According to a specific embodiment, the TBI comprises a single or fractionated irradiation dose on days −2 to −1 (i.e. prior to transplantation).

According to a specific embodiment, the TBI comprises a single or fractionated irradiation dose on day −1 (i.e. one day prior to transplantation).

According to a specific embodiment, the TLI comprises an irradiation dose within the range of 0.5-1 Gy, 0.5-1.5 Gy, 0.5-2.5 Gy, 0.5-5 Gy, 0.5-7.5 Gy, 0.5-10 Gy, 0.5-15 Gy, 1-1.5 Gy, 1-2 Gy, 1-2.5 Gy, 1-3 Gy, 1-3.5 Gy, 1-4 Gy, 1-4.5 Gy, 1-5 Gy, 1-5.5 Gy, 1-6 Gy, 1-7 Gy, 1-1.5 Gy, 1-7.5 Gy, 1-10 Gy, 2-3 Gy, 2-4 Gy, 2-5 Gy, 2-6 Gy, 2-7 Gy, 2-8 Gy, 2-9 Gy, 2-10 Gy, 3-4 Gy, 3-5 Gy, 3-6 Gy, 3-7 Gy, 3-8 Gy, 3-9 Gy, 3-10 Gy, 4-5 Gy, 4-6 Gy, 4-7 Gy, 4-8 Gy, 4-9 Gy, 4-10 Gy, 5-6 Gy, 5-7 Gy, 5-8 Gy, 5-9 Gy, 5-10 Gy, 6-7 Gy, 6-8 Gy, 6-9 Gy, 6-10 Gy, 7-8 Gy, 7-9 Gy, 7-10 Gy, 8-9 Gy, 8-10 Gy, 10-12 Gy, 10-15 Gy, 10-20 Gy, 10-30 Gy, 10-40 Gy, 10-50 Gy, 0.5-20 Gy, 0.5-30 Gy, 0.5-40 Gy or 0.5-50 Gy.

According to a specific embodiment, the TLI comprises a single or fractionated irradiation dose within the range of 1-12 Gy.

According to a specific embodiment, the TLI comprises a single or fractionated irradiation dose within the range of 1-7.5 Gy.

According to a specific embodiment, the TLI comprises a single or fractionated irradiation dose within the range of 1-6 Gy.

According to a specific embodiment, the TLI comprises a single or fractionated irradiation dose within the range of 1-5 Gy.

According to a specific embodiment, the TLI comprises a single or fractionated irradiation dose of 6 Gy.

According to a specific embodiment, the TLI comprises a single or fractionated irradiation dose of 5 Gy.

According to a specific embodiment, the TLI comprises a single or fractionated irradiation dose of 4 Gy.

According to a specific embodiment, the TLI comprises a single or fractionated irradiation dose of 3 Gy.

According to an embodiment, TLI treatment is administered to the subject 1-10 days (e.g. 1-3 days, e.g. 1 day) prior to transplantation. According to one embodiment, the subject is conditioned once with TLI 1, 2, 3 or 4 days (e.g. 1 day) prior to transplantation.

According to a specific embodiment, the TLI comprises a single or fractionated irradiation dose on days −3 to −1 (i.e. prior to transplantation).

According to a specific embodiment, the TLI comprises a single or fractionated irradiation dose on days −2 to −1 (i.e. prior to transplantation).

According to a specific embodiment, the TLI comprises a single or fractionated irradiation dose on day −1 (i.e. one day prior to transplantation).

According to one embodiment, the conditioning comprises a chemotherapeutic agent. Exemplary chemotherapeutic agents include, but are not limited to, Busulfan, Busulfex, Cyclophosphamide, Everolimus, Fludarabine, Melphalan, Myleran, Trisulphan, and Thiotepa.

According to one embodiment, the conditioning comprises an immunosuppressant agent, such as but not limited to, Rapamycin.

The chemotherapeutic agent/s and/or immunosuppressant agent/s may be administered to the subject in a single dose or in several doses e.g. 2, 3, 4, 5, 6, 7, 8, 9, 10 or more doses (e.g. daily doses) prior to or subsequent to transplantation.

According to a specific embodiment, the subject is administered Rapamycin.

Rapamycin (also known as Sirolimus and Rapamune) is commercially available from e.g. Pfizer. Rapamycin analogs include, e.g. CCI-779, RAD001, AP23573.

According to one embodiment, a therapeutically effective amount of Rapamycin comprises 0.01 mg/day/kg to 3 mg/day/Kg ideal body weight, 0.02 mg/day/kg to 1.5 mg/day/Kg ideal body weight, e.g. 0.05 mg/day/kg to 1.0 mg/day/Kg, e.g. 0.1 mg/day/kg to 0.3 mg/day/Kg ideal body weight.

According to one embodiment, a therapeutically effective amount of Rapamycin comprises about 0.1 mg/day/Kg ideal body weight.

According to one embodiment, a therapeutically effective amount of Rapamycin comprises about 0.3 mg/day/Kg ideal body weight.

According to one embodiment, a therapeutically effective amount of Rapamycin comprises about 0.5 mg/day/Kg ideal body weight.

According to one embodiment, a therapeutically effective amount of Rapamycin comprises about 0.7 mg/day/Kg ideal body weight.

According to one embodiment, a therapeutically effective amount of Rapamycin comprises about 1 mg/day/Kg ideal body weight.

According to one embodiment, a therapeutically effective amount of Rapamycin comprises about 2 mg/day/Kg ideal body weight.

According to one embodiment, a therapeutically effective amount of Rapamycin comprises about 2.5 mg/day/Kg ideal body weight.

According to one embodiment, a therapeutically effective amount of Rapamycin comprises about 3 mg/day/Kg ideal body weight.

According to one embodiment, Rapamycin is effected for 3-10 days (e.g. 3, 4, 5 or 6 days). For example, Rapamycin may be administered from day −4 to day +10, e.g. from day −1 to day +4 (e.g. on days −1, 0, +1, +2, +3 and +4 of immature hematopoietic cell transplantation).

According to one embodiment, Rapamycin is not administered for more than 4, 5, 6 or 7 days.

According to a specific embodiment, Rapamycin may be administered prior to, concomitantly with or following the non-GVHD inducing anti-third party Tcm cells (i.e. veto cells, e.g. one day prior to, on the same day, or on the following day from administration of veto cells). Accordingly, according to a specific embodiment, non-GVHD inducing anti-third party Tcm cells (i.e. veto cells) are administered on day 0, i.e. together with the immature hematopoietic cells, and Rapamycin is administered on days −4 to +4 e.g. from day −1 to day +4 (e.g. on days −1, 0, +1, +2, +3 and +4 of immature hematopoietic cell transplantation). According to another specific embodiment, immature hematopoietic cells are administered on day 0, non-GVHD inducing anti-third party Tcm cells (i.e. veto cells) are administered on day +7, and Rapamycin is administered on days −4 to +4 e.g. from day −1 to day +4 (e.g. on days −1, 0, +1, +2, +3 and +4 of immature hematopoietic cell transplantation).

According to a specific embodiment, Rapamycin is effected at a dose of 0.3 mg/day/Kg ideal body weight on day −1, and then administered at a dose of 0.1 mg/day/Kg ideal body weight up to day +4 (e.g. on days 0, +1, +2, +3 and +4 of immature hematopoietic cell transplantation).

According to a specific embodiment, the combination of Rapamycin and non-GVHD inducing anti-third party Tcm cells (i.e. veto cells) is used to enable hematopoietic cell transplantation in the absence of graft rejection and GVHD.

According to a specific embodiment, the combination of Rapamycin, non-GVHD inducing anti-third party Tcm cells (i.e. veto cells) and irradiation (e.g. TBI or TLI) is used to enable hematopoietic cell transplantation in the absence of graft rejection and GVHD.

According to a specific embodiment, the transplantation protocol comprises a single or fractionated irradiation dose of 1-7.5 Gy (e.g. 4.5-5 Gy TBI) on day −3 to −1 (e.g. on day −1), immature hematopoietic cells are administered on day 0 (e.g. megadose T cell depleted, e.g. comprising at least about 5×10⁶ CD34⁺ cells per kilogram ideal body weight of the subject), non-GVHD inducing anti-third party Tcm cells (i.e. veto cells) are administered on day 0 to +20, e.g. on day 0-10, e.g. on day +7 (e.g. at a dose of at least about 0.5×10⁶/Kg ideal body weight, e.g. at a dose of 2.5-10×10⁶ CD8⁺ cells per kg ideal body weight, e.g. 5×10⁶ CD8⁺ cells per kg ideal body weight), and Rapamycin is administered on days −4 to +4 e.g. from day −1 to day +4 (e.g. on days −1, 0, +1, +2, +3 and +4 of immature hematopoietic cell transplantation) at a dose of about 0.1-3 mg/day/Kg ideal body weight.

According to one embodiment, the conditioning comprises in vivo T cell debulking.

According to some embodiments, the in-vivo T cell debulking is effected by antibodies.

According to some embodiments, the in-vivo T cell debulking is effected prior to TBI or TLI.

According to some embodiments of the invention, the antibodies comprise an anti-CD8 antibody, an anti-CD4 antibody, or both.

According to some embodiments of the invention, the antibodies comprise anti-thymocyte globulin (ATG) antibodies, anti-CD52 antibodies or anti-CD3 (OKT3) antibodies.

Anti-thymocyte globulin (ATG) antibodies are commercially available from e.g. Genzyme and Pfizer, e.g. under the brand names e.g. Thymoglobulin and Atgam.

According to a specific embodiment, the subject is not treated with ATG prior to transplantation.

It will be appreciated that when using no ATG or lower doses of ATG are used, e.g. single dose or two doses (e.g. each at a dose of about 2 mg per kg ideal body weight), higher radiation doses can be used as part of the non-myeloablative conditioning protocol (e.g. TBI at a single or fractionated irradiation dose of 3-5 Gy, e.g. 3 Gy, 4 Gy or 5 Gy).

According to a specific embodiment, the subject is administered Cyclophosphamide.

According to one embodiment, the method comprises post-transplant administration of cyclophosphamide.

According to one embodiment, cyclophosphamide is administered to the subject 1, 2, 3, 4, 5 days post-transplant (i.e., D+1, +2, +3, +4, +5).

According to a specific embodiment, cyclophosphamide is administered to the subject in two doses 3 and 4 days post-transplant.

According to one embodiment, the present invention further contemplates administration of cyclophosphamide prior to transplantation (e.g. on days 6, 5, 4 or 3 prior to transplantation, i.e. D-6 to −3) in addition to the administration following transplantation.

For example, in case of immature hematopoietic cell transplantation, the therapeutic effective amount of cyclophosphamide comprises about 1-25 mg, 1-50 mg, 1-75 mg, 1-100 mg, 1-250 mg, 1-500 mg, 1-750 mg, 1-1000 mg, 5-50 mg, 5-75 mg, 5-100 mg, 5-250 mg, 5-500 mg, 5-750 mg, 5-1000 mg, 10-50 mg, 10-75 mg, 10-100 mg, 10-250 mg, 10-500 mg, 10-750 mg, 10-1000 mg, 25-50 mg, 25-75 mg, 25-100 mg, 25-125 mg, 25-200 mg, 25-300 mg, 25-400 mg, 25-500 mg, 25-750 mg, 25-1000 mg, 50-75 mg, 50-100 mg, 50-125 mg, 50-150 mg, 50-175 mg, 50-200 mg, 50-250 mg, 50-500 mg, 50-1000 mg, 75-100 mg, 75-125 mg, 75-150 mg, 75-250 mg, 75-500 mg, 75-1000 mg, 100-125 mg, 100-150 mg, 100-200 mg, 100-300 mg, 100-400 mg, 100-500 mg, 100-1000 mg, 125-150 mg, 125-250 mg, 125-500 mg, 125-1000 mg, 150-200 mg, 150-300 mg, 150-500 mg, 150-1000 mg, 200-300 mg, 200-400 mg, 200-500 mg, 200-750 mg, 200-1000 mg, 250-500 mg, 250-750 mg, 250-1000 mg per kilogram ideal body weight of the subject.

According to a specific embodiment, the therapeutic effective amount of cyclophosphamide is about 25-200 mg per kilogram ideal body weight of the subject.

According to one embodiment, cyclophosphamide is administered in a single dose.

According to one embodiment, cyclophosphamide is administered in multiple doses, e.g. in 2, 3, 4, 5 doses or more.

According to a specific embodiment, cyclophosphamide is administered in two doses.

According to a specific embodiment, cyclophosphamide is not administered in more than 1, 2, 3, 4 or 5 doses (e.g. over 1, 2, 3, 4 or 5 days).

The dose of each cyclophosphamide administration may comprise about 5 mg, 7.5 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 350 mg, 400 mg, 450 mg or 500 mg per kilogram ideal body weight of the subject.

According to a specific embodiment, each dose of cyclophosphamide is 50 mg per kilogram ideal body weight of the subject.

Cyclophosphamide is commercially available from e.g. Zydus (German Remedies), Roxane Laboratories Inc—Boehringer Ingelheim, Bristol-Myers Squibb Co—Mead Johnson and Co, and Pfizer—Pharmacia & Upjohn, under the brand names of Endoxan, Cytoxan, Neosar, Procytox and Revimmune.

According to a specific embodiment, veto cells are administered following Cyclophosphamide (e.g. 2-5 days following Cyclophosphamide). Accordingly, according to a specific embodiment, immature hematopoietic cells are administered on day 0, Cyclophosphamide is administered on days +3 and +4, and veto cells are administered on day +7.

According to a specific embodiment, the subject is administered Fludarabine.

According to one embodiment, Fludarabine is effected for 3-10 days (e.g. 3, 4, 5 or 6 days, e.g. for 4 days). For example, Fludarabine may be administered from day −10 to day −7, e.g. from day −8 to day −5, e.g. from day −6 to −3 (e.g. on days −6, −5, −4, −3 prior to immature hematopoietic cell transplantation).

According to one embodiment, Fludarabine is not administered for more than 3, 4, 5, 6 or 7 days.

According to a specific embodiment, the Fludarabine is administered at a dose of about 5-100 mg/m²/day e.g. 30 mg/m²/day for 3, 4, 5 or 6 consecutive days (e.g. 4 consecutive days) prior to transplantation (e.g. on days −6 to −3).

Fludarabine is commercially available from e.g. Sanofi Genzyme, Bayer and Teva, e.g. under the brand name e.g. Fludara.

According to a specific embodiment, the subject may be treated daily with Fludarabine on days −6 to −3 prior to transplant (e.g. at a dose of about 30 mg/m²), followed by low dose TBI (e.g. single or fractionated irradiation dose of e.g. 1-5 Gy, e.g. 3 Gy) on day −3 to day 0, e.g. on day −1 prior to transplant as the preparative regimen. Immature hematopoietic cells are administered (e.g. infused e.g. by IV) on day 0 (e.g. megadose T cell depleted, e.g. comprising at least about 5×10⁶ CD34⁺ cells per kilogram ideal body weight of the subject). Cyclophosphamide (Cy) is administered in two doses following immature hematopoietic cell transplantation (e.g. on days +3 and +4, at a dose of e.g. 25-100 mg per kilogram ideal body weight of the subject) followed by the infusion of the anti-third party Tcm cells (i.e. veto cells) on day +5 to +10 (e.g. on day +7 after immature hematopoietic cell transplantation), e.g. at a dose of 2.5-10×10⁶ CD8⁺ cells per kg ideal body weight (e.g. 5×10⁶ CD8⁺ cells per kg ideal body weight). Optionally, ATG can be administered daily on days −9 to −7 prior to transplantation so as to induce T cell debulking in the subject (e.g. at a dose of about 2 mg per Kg ideal body weight).

According to a specific embodiment, TBI is administered on the day of transplantation of the T cell depleted immature hematopoietic cells e.g. in the morning of day −1 or day 0, and transplantation is carried out on the same day, e.g. in the evening of day −1. According to a specific embodiment, a second dose of T cell depleted immature hematopoietic cells is carried out the following day (e.g. on day 0).

According to a specific embodiment, the T cell depleted immature hematopoietic cells comprise fresh cells.

According to a specific embodiment, the T cell depleted immature hematopoietic cells comprise cells which were previously obtained, cryopreserved and thawed on the day of the transplant).

According to one embodiment, the subject is treated with additional supportive drugs, e.g. chemotherapy adjuvants.

According to one embodiment, the subject is treated with a dose of Mesna (e.g. 10 mg/kg intravenous piggy back (IVPB) just prior to the first dose of cyclophosphamide (e.g. 2 hours, 1 hour, 30 minutes, 15 minutes prior to the first dose of cyclophosphamide). According to one embodiment, administration of mesna is repeated every 4 hours for a total of 10 doses.

Mesna is commercially available from e.g. Baxter under the brand names of Uromitexan and Mesnex.

According to a one embodiment, the subject is treated with ondansetron (or another anti-emetic) prior to each dose of Cyclophosphamide (Cy).

According to one embodiment, the subject is not treated with an immunosuppressive agent (e.g. aside from the rapamycin and veto cells, or cyclophosphamide and veto cells, as discussed herein).

According to a specific embodiment, the subject is not treated with long term GVHD prophylaxis (e.g. immunosuppressive agent), e.g. for more than 7-14 days post-transplant, e.g. 7, 8, 9, 10, 11, 12, 13 or 14 days post-transplant.

According to one embodiment, the subject is treated with an immunosuppressive agent.

Examples of immunosuppressive agents include, but are not limited to, Tacrolimus (also referred to as FK-506 or fujimycin, trade names: Prograf, Advagraf, Protopic), Mycophenolate Mofetil, Mycophenolate Sodium, Prednisone, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE), etanercept, TNF.alpha. blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, tramadol. These agents may be administered individually or in combination.

According to one embodiment, corticosteroids are not administered as a pretreatment to the veto cells.

It is expected that during the life of a patent maturing from this application many relevant non-myeloablative conditioning agents will be developed and the scope of the term non-myeloablative conditioning agents is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

Animals

Mice used were 6- to 12-week-old mice of the following strains: C57BL/6 (H-2^{b}), FVB (H-2^{q}) were purchased from Harlan Israel (Rehovot, Israel). B6 nude (Foxn1nu/J, H-2^{b}) and β-actin-GFP (on C57BL/6J background) mice were purchased from Jackson laboratories and bred in the Weizmann institute animal facility. NOD/LtJ (H-2K^{d}/H-2D^{b}) mice are bred in the Weizmann institute animal facility. All mice were kept in small cages and fed sterile food and acid water in a SPF environment under conditions approved by the Institutional Animal Care and Use Committee at the Weizmann Institute.

Preparation of Host Non-Reactive Donor Anti-Third-Party Veto Cells

Anti-3rd party Tcm with a central memory phenotype (CD8⁺CD44⁺CD62L⁺) were prepared as previously described [Ophir E. at al., Blood (2010) 115(10): 2095-104]. Briefly, splenocytes of donor mice were cultured against irradiated third-party splenocytes for 60 hours under cytokine deprivation. Subsequently, CD8 cells were positively selected using magnetic particles (BD Pharmingen) and cultured in an Ag-free environment. rhIL-15 (20 ng/mL; R&D Systems) was added every second day. To attain a purified population, at the end of the culture (day 16), cells were positively selected for CD62L expression (magnetic-activated cell sorting—MACS® Cell Separation, Miltenyi Biotec) and cells were retrieved for FACS analysis.

Bone Marrow Preparation

Long bones were harvested from B6 nude (Foxn1nu/J, H-2^{b}) mice (7-12 weeks of age). Bone marrow was extracted by grinding the bones to reach a single cell suspension. Bone marrow was counted and brought to the correct concentration and was then injected to mice i.v. to the tail vein.

Reduced Conditioning Model for BM Allografting

NOD/LtJ (H-2K^{d}/H-2D^{b}) female mice (8 weeks of age) received sublethal TBI at the indicated doses (e.g. 4.5 Gy) on day −1. On the following day (e.g. day 0), animals were transplanted with a “megadose” transplant of B6 nude (Foxn1nu/J, H-2^{b}) BM cells at (25×10⁶) with or without 5×10⁶ anti 3^{rd}-party veto cells (C57BL/6 (H-2^{b}) Tcm). Hosts also received subcutaneous injections (days −1 to +4) of 12.5 μ/mice/day of rapamycin (Rapamune). Post transplantation, mice were evaluated weekly for overall appearance, weight and were bled to test blood glucose levels to detect hyperglycemia. Sickness was defined when the measured blood glucose levels were above 200 mg/dl in two consecutive measurements. Chimerism analysis was conducted periodically (every 30 days).

Chimerism Analysis

Chimerism analysis was conducted periodically and determined by flow cytometry. Peripheral blood was collected by retro-orbital bleeding. Cells were fractionated on Ficoll-Paque (Amersham Pharmacia Biotech, AB), and the isolated mononuclear cells of each mouse were double-stained by direct immuno-fluorescence labeled antibodies against donor and host surface markers. Fluorescence-activated cell sorting (FACS) analysis was performed using a modified Becton Dickinson FACSCanto I. Cells were stained with labeled antibodies specific for: CD8a, CD4, CD3, CD44, CD62L, H2Dd, H2Kb, (Biolegend; BD; Miltenyi). Data were analyzed using FACSDiva 8.0. software and FlowJo v10.2 software.

Flow Cytometry Analysis

Fluorescence-activated cell sorting (FACS) analysis was performed using a modified Becton Dickinson FACSCanto I. Cells were stained with labeled antibodies specific for: CD3-PE/FITC/APC, CD4-APC/PB, CD8α-PE/FITC/APC/APC-Cy7, CD19-PE/FITC/APC/PE-Cy7, CD62L-PE/FITC/APC, CD44-PE/FITC/APC, CD45-APC-Cy7, CD11b-PeCy7, Ly6G-PB, Ly6C-PerCP, H2K^{b}-PE/FITC, H2D^{d}-PE/FITC (BD Pharmigen; Biolegend; Miltenyi). Data was analyzed using FACSDiva 8.0. software and FlowJo v10.2 software.

TCR Library Preparation

Spleens, harvested from mice in all experimental groups, were processed and cells were isolated by density-gradient centrifugation using Ficoll-Paque PLUS (GE Healthcare Lifescience). Thereafter, positive selections using Miltenyi beads (magnetic-activated cell sorting—MACS® Cell Separation, Miltenyi Biotec,) were performed according to manufacturers' instructions. CD8 positive selection was performed first, followed by CD4 positive selection from CD8 negative fraction, followed by CD25 positive selection from CD4 negative fraction. Sorted cell populations were lysed separately and RNA stabilized in RLT buffer (Qiagen) following manufacturers' instructions. RNA was isolated using an RNeasy extraction kit following the manufacturers' instructions (Qiagen) and stored at −80° C. The library preparation was based on a protocol for quantitative TCR sequencing. Full details of the method have been previously published [Oakes T et al. Front Immunol. (2017), 8:1267]. Briefly, high-quality total RNA from a population of T cells was quantified. Then, each sample was treated with DNase, and taken to reverse transcription for cDNA production. Next—ligation of primers and Unique molecular identifiers (UMIs) to the V constant region, and amplification PCR. Finally, samples were purified and taken to sequencing. Data was assessed using pipeline analysis.

Image Acquisition by Two-Photon Laser Scanning Microscopy

NOD mice were transplanted according to the protocol elaborated above with B6 nude (Foxn1nu/J, H-2^{b}) BM cells (25×10⁶) and 5×10⁶ anti 3^{rd}-party GFP positive on C57BL/6 background (H-2^{b}) Tcm. Hosts were sacrificed one year post transplantation and organs were harvested and observed by microscope.

A Zeiss LSM 880 upright microscope fitted with a Coherent Chameleon Vision laser was used for intravital imaging experiments. Images were acquired with a femtosecond-pulsed two-photon laser tuned to 940 nm. The microscope was fitted with a filter cube containing 565 LPXR to split the emission to a PMT detector (with a 579-631-nm filter for tdTomato fluorescence) and to an additional 505 LPXR mirror to further split the emission to 2 GaAsp detectors (with a 500-550-nm filter for GFP fluorescence). GC LZ (labeled by NP-tdTomato) or DZ (facing the paracortex and containing autofluorescent macrophages) in the popliteal LN were photoactivated at 820 nm. Tile images were acquired as 100-200-μm Z-stacks with 5-μm steps between each Z-plane. The zoom was set to 1.5, and pictures were acquired at 512×512 x-y resolution.

Statistical Analysis

The analysis of survival data was performed using Kaplan-Meier curves (log-rank test). Comparison of means was conducted using the Student t test. P values<0.05 were considered statistically significant.

### Example 1

### Treatment of T Cell Mediated Diabetes Using Veto Tcm Cells and T Cell Depleted Bone Marrow Transplant Following a Reduced Intensity Conditioning

In this study, Tcm veto cells were generated from splenocytes obtained from C57BL/6 donors (H-2^{b}) cultured against irradiated third-party splenocytes (FVB; H-2^{q}), under cytokine deprivation. The selective expansion of CD8 mouse T cells against 3rd party stimulators under these conditions leads to selective ‘death by neglect’ of bystander anti-host T cell clones that could mediate graft versus host disease (GVHD); such cells are further diluted out by subsequent expansion of anti-3rd party T clones during further culture in the presence of IL-15. In addition to causing selective loss of GVH reactive T cells, these culture conditions induce a central memory phenotype shown to be crucial for robust veto activity in vivo.

To evaluate the safety and efficacy of such a transplant for diabetes therapy, 8 week old NOD (H-2^{d}) mice (before diabetes onset, i.e. before developing symptoms of diabetes) were treated with 4.5 Gy TBI conditioning at day −1, followed by anti-3rd party veto Tcm and megadose nude bone marrow (NuBM) on day 0 (FIG. 1). In order to reduce the conditioning toxicity, short term treatment with Rapamycin was used which was previously found to enable chimerism induction when combined with veto cells following 4.5 TBI [Ophir E et al, Blood (2013) 121(7):1220-1228]. Accordingly, Rapamycin treatment was affected from day −1 to day +4 (FIG. 1). Controls were untreated or received conditioning with no transplant. In four independent experiments (n=35 NOD mice in the transplanted group), high chimerism from 83.5% to 99.6% was found in all transplanted mice at 6 months post-transplant. These results were consistent in 4 independent experiments. Notably, mice that did not achieve chimerism, died before that time-point due to diabetes development (FIG. 3A).

Subsequently, a more profound evaluation of the nature of chimerism was performed by FACS analysis demonstrating multi-lineage chimerism, in various cell types including lymphocytes, myeloid cells, dendritic cells, etc. (FIGS. 3B-J).

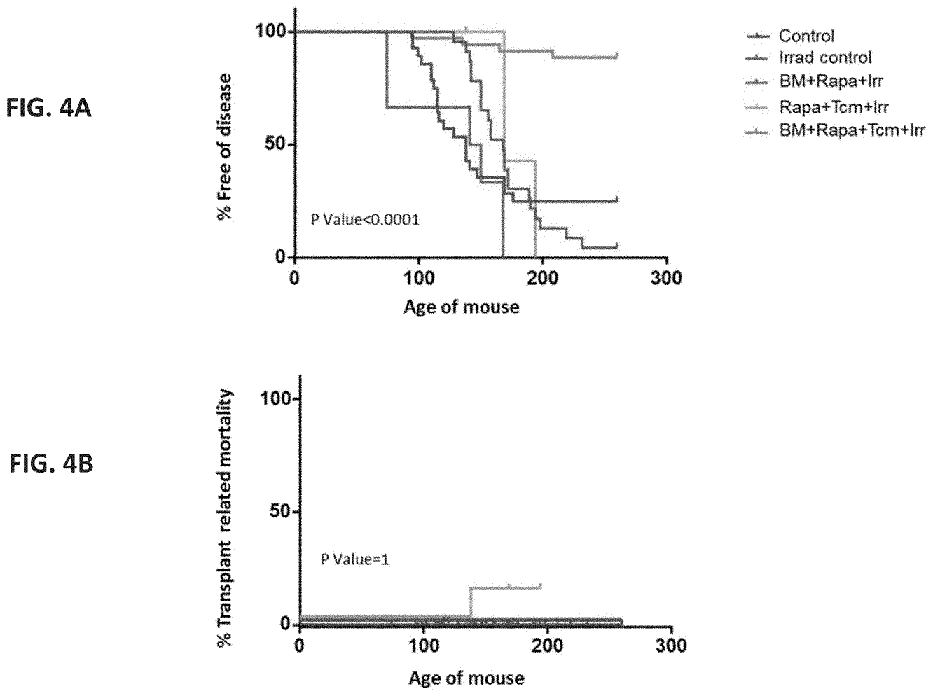

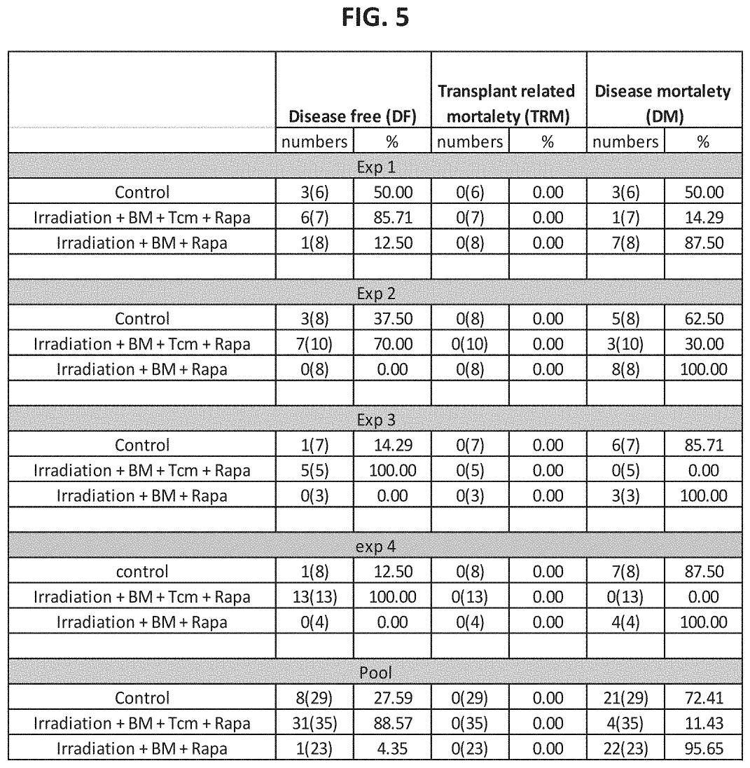

With a follow up of 250 days, 72.4% mice in the control group, with no treatment, and 95% of the conditioned group (receiving TBI and Rapamycin only) died of diabetes, while only 8.5% or 11.4% diabetes-related mortality was observed in the mice transplanted with full conditioning and Tcm administration (FIGS. 2A and 4A, respectively). In contrast, 88.5% of the treatment group survived and did not develop diabetes (FIG. 5). Furthermore, no GVHD or other transplant-related mortality was observed in the transplanted mice (FIGS. 2B and 4B) strongly confirming the safety of this treatment modality.

These results demonstrate a proof of concept for the safety and efficacy of non-myeloablative allogeneic TD-HSCT in type 1 diabetes.

### Example 2

### Veto Tcm Persistence in Transplanted NOD Mice

The question of Tcm survival in recipient NOD mice is of major interest for evaluation of tolerance persistence as well as for future applications of these veto cells. To that end, Tcm veto cells were generated from GFP mice (C57BL background). Cells were transplanted according to the same protocol described in FIG. 1 (e.g. 4.5 Gy irradiation, Rapamycin treatment from day −1 to day +4, and megadose B6 nude bone marrow along with veto cells on day 0. One-year post transplantation, mice were sacrificed, and lymphoid organs were evaluated by Two-photon laser scanning microscopy. As shown in FIGS. 6A-D, in three evaluated mice, GFP+ Tcm cells were present in the spleen and in several types of lymph nodes.

### Example 3

### T Cell Repertoire Analysis of Diabetes Prevention in Chimeric NOD Mice Supports a Deletion-Based Mechanism

Accordingly, T cell repertoire analysis was performed in search for the mechanism underlying the prevention of diabetes development. Comparison between the variable complementary-determining regions (CDR3) in the T cell receptors of mice demonstrated higher sharing level between amino acids in the young non-treated mice as shown in FIG. 7A. In contrast, the treated mice had a lower sharing level of sequences.

Evaluation of the Vβ usage revealed different usage between CD4 and CD8 as expected. Notably, CD8 Vβ usage of treated mice was different from CD8 Vβ usage in the non-treated mice (FIG. 7B). Different Vβ usage of CD4 and CD25 was also seen when comparing treated to non-treated mice.

Next, known CDR3 sequences connected to type I diabetes mellitus [Gioia L. et al. Sci Immunol (2019) 4(38):10; Baker R L et al. Diabetes (2018) 67(9):1836-1846] were searched and evaluated for their presence in the current data. As shown in FIG. 8, sequences related to Insulin or GAD 65, that represent T cell receptors that are directed against these components, are found in NOD mice, before and after development of diabetes. Notably, these sequences were not found in the treated mice strongly indicating deletion of these clones by the allogeneic bone marrow transplant protocol.

### Example 4

Protocol for Human Treatment

To evaluate a treatment protocol for human correction of T cell mediated autoimmune diseases, a reduced intensity conditioning regimen comprising Anti-thymocyte globulin (ATG), Fludarabine (Fu) and low dose total body irradiation (TBI) is contemplated. The regimen further comprises megadose T cell depleted bone marrow, post-transplant cyclophosphamide and veto cells.

The treatment protocol is as follows:

−9 ATG (Thymoglobulin) 2 mg per kg ideal body weight

−8 ATG (Thymoglobulin) 2 mg per kg ideal body weight

−7 ATG (Thymoglobulin) 2 mg per kg ideal body weight

−6 Fludarabine 30 mg/m²

−5 Fludarabine 30 mg/m²

−4 Fludarabine 30 mg/m²

−3 Fludarabine 30 mg/m²

−2 Rest (no treatment)

−1 TBI 3 Gy in a single fraction.

(−1 optionally first infusion of megadose T cell depleted immature hematopoietic cells 6-16 hours after TBI)

0 infusion of megadose T cell depleted immature hematopoietic cells

(i.e. the subject is administered with at least 5×10⁶ CD34⁺ cells per kilogram ideal body weight, in one or two infusions)

+3 Cyclophosphamide (CY) 50 mg/kg ideal body weight/day IV

+4 Cyclophosphamide (CY) 50 mg/kg ideal body weight/day IV

+7 infusion of anti-third party Tcm cells (i.e. veto cells) at doses of 5×10⁶-10×10⁶ CD8⁺ cells per kg ideal body.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of treating or preventing a T cell mediated autoimmune disease in a subject in need thereof, the method comprising:
(a) transplanting immature hematopoietic cells into the subject; and
(b) administering to the subject a therapeutically effective amount of an isolated population of non-GVHD inducing anti-third party cells comprising cells having a central memory T-lymphocyte (Tcm) phenotype, said cells being tolerance inducing cells and capable of homing to the lymph nodes following transplantation, thereby treating the T cell mediated autoimmune disease in the subject.

2. The method of claim 1, wherein said isolated population of non-GVHD inducing anti-third party cells are:
(i) co-administrated with said immature hematopoietic cell transplant;
(ii) administered following said immature hematopoietic cell transplant;
(iii) administered on day 1-20 following said immature hematopoietic cell transplant; and/or
(iv) administered at a dose of at least 0.5×10⁶ CD8⁺ cells per kg ideal body weight.

3. The method of claim 1, wherein said isolated population of non-GVHD inducing anti-third party cells are used for reducing graft rejection and/or inducing donor specific tolerance.

4. The method of claim 1, wherein said isolated population of non-GVHD inducing anti-third party cells are generated by a method comprising:
(a) contacting peripheral blood mononuclear cells (PBMCs) with a third party antigen or antigens in a culture deprived of cytokines so as to allow enrichment of antigen reactive cells; and
(b) culturing said cells resulting from step (a) in the presence of cytokines so as to allow proliferation of cells comprising said central memory T-lymphocyte (Tcm) phenotype, thereby generating the non-GVHD inducing anti-third party cells.

5. The method of claim 4, further comprising:
(i) depleting CD4+ and/or CD56+ expressing cells from said PBMCs prior to said contacting with said third party antigen or antigens;
(ii) selecting CD45RA⁺ expressing cells so as to obtain a population of naïve T cells expressing a CD45RA⁺CD8⁺ phenotype;
(iii) depleting CD45RA⁺ expressing cells so as to obtain a population enriched of memory T cells expressing a CD45RA⁻CD8⁺ phenotype; and/or
(iv) selecting for CD3⁺, CD8⁺, CD62L⁺, CD45RA⁻, CD45RO⁺ signature.

6. The method of claim 4, wherein:
(i) said contacting with said antigen or antigens of step (a) in effected in the presence of IL-21;
(ii) said culturing said cells resulting from step (a) in the presence of cytokines comprises culturing said cells in the presence of IL-15; and/or
(iii) said culturing said cells resulting from step (a) in the presence of cytokines comprises culturing said cells in the presence of IL-21, IL-15 and/or IL-7.

7. The method of claim 4, wherein said antigen or antigens:
(i) is selected from the group consisting of a viral antigen, a bacterial antigen, a tumor antigen, an autoimmune disease related antigen, a protein extract, a purified protein and a synthetic peptide;
(ii) is presented by autologous antigen presenting cells, non-autologous antigen presenting cells, artificial vehicles or artificial antigen presenting cells;
(iii) is presented by antigen presenting cells of the same origin as said PBMCs; and/or
(iv) comprises stimulatory cells selected from the group consisting of cells purified from peripheral blood lymphocytes, spleen or lymph nodes, cytokine-mobilized PBLs, in vitro expanded antigen-presenting cells (APC), in vitro expanded dendritic cells and artificial antigen presenting cells.

8. The method of claim 1, wherein said immature hematopoietic cells comprise:
(i) T cell depleted immature hematopoietic cells;
(ii) at least 5×10⁶ CD34⁺ cells per kilogram ideal body weight of the subject;
(iii) are depleted of CD3⁺ and/or CD19⁺ expressing cells;
(iv) less than 5×10⁵ CD3⁺ expressing cells per kg ideal body weight of the subject; and/or
(v) are non-syngeneic with the subject.

9. The method of claim 1, wherein said immature hematopoietic cells and said isolated population of non-GVHD inducing anti-third party cells are obtained from the same donor.

10. The method of claim 1, further comprising conditioning the subject under non-myeloablative conditioning.

11. The method of claim 10, wherein said non-myeloablative conditioning comprises at least one of total body irradiation (TBI), a partial body irradiation (TLI), a chemotherapeutic agent, an antibody immunotherapy or a co-stimulatory blockade.

12. The method of claim 11, wherein said TBI:
(i) comprises an irradiation dose within the range of 1-6 Gy;
(ii) is to be affected on any one of days −3 to day 0 of said transplanting; and/or
(iii) is to be affected one or two days prior to said transplanting.

13. The method of claim 11, wherein said chemotherapeutic agent comprises at least one of Everolimus, Fludarabine, Cyclophosphamide, Busulfan, Trisulphan, Melphalan or Thiotepa.

14. The method of claim 1, further comprising administering to the subject a therapeutically effective amount of Rapamycin.

15. The method of claim 14, wherein said therapeutically effective amount of Rapamycin comprises at least 0.1 mg Rapamycin per day per kilogram ideal body weight of the subject.

16. The method of claim 14, wherein said Rapamycin is to be administered to the subject on days −1 to +4 of said transplanting.

17. The method of claim 10, wherein said non-myeloablative conditioning comprises T cell debulking.

18. The method of claim 17, wherein said T cell debulking is effected by at least one of anti-thymocyte globulin (ATG) antibodies, anti-CD52 antibodies or anti-CD3 (OKT3) antibodies.

19. The method of claim 10, wherein said non-myeloablative conditioning comprises a therapeutically effective amount of Fludarabine.

20. The method of claim 1, further comprising administering to the subject a therapeutically effective amount of cyclophosphamide.

21. The method of claim 20, wherein said therapeutically effective amount of cyclophosphamide comprises 25-200 mg per kilogram ideal body weight of the subject.

22. The method of claim 20, wherein said cyclophosphamide is to be administered to the subject on days +3 and +4 of said transplanting.

23. The method of claim 1, the method comprising:
(a) conditioning the subject under non-myeloablative conditioning, wherein said non-myeloablative conditioning comprises a total body irradiation (TBI) and a immunosuppressive agent, wherein said TBI and said immunosuppressive agent are administered on days −4 to +4 of transplantation;
(b) transplanting into the subject a dose of T cell depleted immature hematopoietic cells, wherein said T cell depleted immature hematopoietic cells comprises at least 5×10⁶ CD34⁺ cells per kilogram ideal body weight of the subject; and
(c) administering to the subject a therapeutically effective amount of an isolated population of non-GVHD inducing anti-third party cells comprising cells having a central memory T-lymphocyte (Tcm) phenotype, said cells being tolerance inducing cells and capable of homing to the lymph nodes following transplantation.

24. The method of claim 1, the method comprising:
(a) conditioning the subject under non-myeloablative conditioning, wherein said non-myeloablative conditioning comprises a total body irradiation (TBI) and a chemotherapeutic agent, wherein said TBI and said chemotherapeutic agent are administered on days −6 to 0 of transplantation;
(b) transplanting into the subject a dose of T cell depleted immature hematopoietic cells, wherein said T cell depleted immature hematopoietic cells comprises at least 5×10⁶ CD34⁺ cells per kilogram ideal body weight of the subject;
(c) administering to the subject a therapeutically effective amount of cyclophosphamide, wherein said therapeutically effective amount of said cyclophosphamide comprises 25-200 mg cyclophosphamide per kilogram ideal body weight of the subject, and wherein said therapeutically effective amount of said cyclophosphamide is to be administered to the subject in two doses on days +3 and +4 following said transplantation of said T cell depleted immature hematopoietic cells; and
(d) administering to the subject a therapeutically effective amount of an isolated population of non-GVHD inducing anti-third party cells comprising cells having a central memory T-lymphocyte (Tcm) phenotype, said cells being tolerance inducing cells and capable of homing to the lymph nodes following transplantation, wherein said isolated population of non-GVHD inducing cells are administered on day +5 to +10 following said transplantation of said T cell depleted immature hematopoietic cells.

25. The method of claim 1, wherein the T cell mediated autoimmune disease is selected from the group consisting of type 1 diabetes mellitus (T1DM), Hashimoto's thyroiditis, multiple sclerosis (MS), rheumatoid arthritis (RA), ankylosing spondylitis, Crohn's disease, ulcerative colitis (UC), non-infectious uveitis, Lupus erythematosus (SLE), Sjogren's syndrome, primary biliary cirrhosis, autoimmune hepatitis, Immune Thrombocytic Purpura (ITP), Chronic Glomerulonephritis, Myasthenia gravis, Systemic Scleroderma, Polymyositis and Addison's disease.

26. The method of claim 25, wherein when the T cell mediated autoimmune disease is type 1 diabetes mellitus (T1DM), the transplant further comprises a pancreatic cell or tissue transplant.

27. The method of claim 26, wherein said pancreatic cell or tissue transplant is from the same donor as said immature hematopoietic cell transplant and/or said isolated population of non-GVHD inducing anti-third party cells.

28. The method of claim 25, wherein when the T cell mediated autoimmune disease is a primary biliary cirrhosis or an autoimmune hepatitis, the transplant further comprises a hepatic cell or tissue transplant.

29. The method of claim 28, wherein said hepatic cell or tissue transplant is from the same donor as said immature hematopoietic cell transplant and/or said isolated population of non-GVHD inducing anti-third party cells.

30. The method of claim 1, wherein the subject is a human subject.
